(19) United States
(12) Patent Application Publication
(10) Pub. No.: US 2018/0305731 A1
(43) Pub. Date: Oct. 25, 2018

(54) CONSERVED CHAPERONE DOMAIN FOR TYPE VI SECRETION SYSTEM

(71) Applicant: UTI LIMITED PARTNERSHIP, Calgary (CA)

(72) Inventors: Tao DONG, Calgary (CA); Xiaoye LIANG, Calgary (CA)

(73) Assignee: UTI Limited Partnership, Calgary, AB (CA)

(21) Appl. No.: 15/738,113

(22) PCT Filed: Jun. 30, 2016
(86) PCT No.: PCT/IB2016/053910

§ 371 Date: Dec. 19, 2017

Related U.S. Application Data
(60) Provisional application No. 62/187,149, filed on Jun. 30, 2015.

(51) Int. Cl. C12Q 1/18; C07K 14/195; C12N 15/90; G01N 33/569

(52) U.S. Cl. CPC C12Q 1/18; C07K 14/195; A61K 38/00; G01N 33/56911; C12N 15/902

(57) ABSTRACT

The present invention provides a method for identifying a T6SS effector as well as the corresponding T6SS effector immunity protein. The present invention also provides a composition and uses there of that include T6SS effector and T6SS effector immunity protein that are identified using the method of the invention. In particular, the method of the invention utilizes a conserved domain sequence of T6SS of Gram-negative bacteria to identify a T6SS effector and its corresponding immunity protein.

### CROSS-REFERENCE TO RELATED APPLICATIONS

This application claims the priority benefit of U.S. Provisional Application No. 62/187,149, filed Jun. 30, 2015, which is incorporated herein by reference in its entirety.

### FIELD OF THE INVENTION

The present invention relates to a method for identifying a T6SS effector as well as the corresponding T6SS effector immunity protein. The present invention also relates to a composition and uses there of that include T6SS effector and T6SS effector immunity protein that are identified using the method of the invention.

### BACKGROUND OF THE INVENTION

The type VI secretion system (T6SS) is a mechanism used by gram-negative bacterial species in injecting effector proteins and virulence factors (such as proteins, toxins, or enzymes) from across the interior (cytoplasm or cytosol) of a bacterial cell into a target cell. The T6SS is often used by gram-negative bacteria to kill eukaryotic predators or prokaryotic competitors. Killing by the T6SS results from repetitive delivery of toxic effectors.

Despite their importance in dictating bacterial fitness, systematic prediction of T6SS effectors remains challenging due to high effector diversity and the absence of a conserved signature sequence. More significantly, without being bound by any theory, it is believed that each T6SS effector has a counter T6SS effector immunity protein that can be used to treat gram-negative bacteria infection in a subject.

Therefore, there is need for a method to identify T6SS effectors in gram-negative bacteria as well as a method for identifying the corresponding T6SS effector immunity protein that can be used to treat gram-negative bacteria.

### SUMMARY OF THE INVENTION

Disclosed herein is a class of T6SS effector chaperone (TEC) proteins that are required for effector delivery through binding to VgrG and effector proteins. The TEC proteins share a highly conserved domain (DUF4123) and are genetically encoded upstream of their cognate effector genes. Using the conserved TEC domain sequence, a large family of TEC genes coupled to putative T6SS effectors were identified in Gram-negative bacteria. This approach was validated by verifying a predicted effector TseC in Aeromonas hydrophila. Other Gram-negative bacteria effectors found using the method of the invention are listed in Tables 1 and 2 below.

TABLE 1

| | | Predicted Function of |
|---|---|---|
| Species | Downstream gene | downstream gene |
| Shewanella woodyi (strain ATCC 51908) | Swoo_2508 | TC toxin, toxin |
| Marinobacter aquaeolei (strain ATCC | Maqu_2577 | TC toxin, toxin |
| 700491) | | |
| | Maqu_2563 | TC toxin, toxin |
| | Maqu_2570 | TC toxin |
| | Maqu_2149 | Cell wall hydrolyses, Autolysin |
| Vibrio fischeri (strain MJ11) | VFMJ11_1492 | lipoprotein |
| | VFMJ11_1311 | Colicin |
| Aeromonas hydrophila ATCC7966 | AHA_1121 | Hydrolase |
| Xanthomonas oryzae pv. oryzae (strain | WP_042465805 | Restriction endonuclease fold |
| MAFF 311018) | | toxin 5 |
| | XOO_4350 | Restriction endonuclease fold |
| | | toxin 5 |
| Pectobacterium carotovorum subsp. | PC1_3233 | TC toxin |
| carotovorum (strain PC1) | | |
| Photorhabdus asymbiotica subsp. | PAU_02483 | triacylglycerol lipase |
| asymbiotica (strain ATCC 43949 | | |
| | WP_015833494 | Restriction endonuclease fold |
| | | toxin 5 |
| Dickeya dadantii (strain Ech586) | Dd586_1279 | TC toxin |
| Xenorhabdus bovienii (strain SS-2004) | XBJ1_0179 | triacylglycerol lipase |
| | XBJ1_1503 | Triacylglycerol lipase |
| Pseudomonas syringae pv. tomato | PSPTO_3485 | lipase |
| (strain DC3000 | | |
| | PSPTO_5438 | TC toxin |
| | PSPTO_2535 | Rtx toxin |
| Pseudomonas fluorescens (strain Pf0-1) | Pfl01_2042 | tRNA nuclease |
| Herbaspirillum seropedicae (strain | WP_041311537 | Restriction endonuclease fold |
| SmR1) | | toxin 5 |
| Photorhabdus luminescens subsp. | WP_011148677 | Restriction endonuclease fold |
| laumondii (strain TT01) | | toxin 5 |
| Burkholderia pseudomallei (strain | YP_108010 | Restriction endonuclease fold |
| K96243) | | toxin 5 |
| Shewanella woodyi (strain ATCC 51908/ | WP_041418147 | tRNA nuclease |
| MS32) | | |
| Psychromonas sp. CNPT3 | WP_015465492 | esterase-lipase |
| Xanthomonas oryzae pv. oryzicola | XOC_RS05770 | phospholipase effector, |
| (strain BLS256) | | hydrolase |
| | WP_041182987 | Tox-REase-5: Restriction |
| | | endonuclease fold toxin 5 |
| | XOC_RS05805 | phospholipase effector, |
| | | hydrolase |
| | XOC_RS06760 | phospholipase effector, |
| | | hydrolase |
| Xanthomonas oryzae pv. oryzae (strain | XOO_RS07065 | phospholipase effector, |
| KACC10331/KXO85) | | hydrolase |
| | XOO_RS22045 | Tox-REase-5 Restriction |
| | | endonuclease fold toxin 5 |
| | XOO_RS17290 | phospholipase effector, |
| | | hydrolase |
| Proteus mirabilis (strain HI4320) | PMI_RS01010 | triacylglycerol lipase |
| | PMI_RS06410 | triacylglycerol lipase |
| Photorhabdus asymbiotica subsp. | PAU_RS14935 | phospholipase |
| asymbiotica (strain ATCC 43949/3105- | | |
| 77) | | |
| Pectobacterium atrosepticum | ECA_RS16870 | TC toxin, toxin |
| | ECA_RS17150 | phospholipase effector, |
| | | hydrolase |
| | ECA_RS20495 | phospholipase effector, |
| | | hydrolase |
| | ECA_RS16870 | TC toxin, toxin |
| Rahnella aquatilis (strain ATCC 33071/ | RAHAQ2_RS22125 | Tox-REase-5 Restriction |
| DSM 4594/JCM 1683/NBRC 105701/ | | endonuclease fold toxin 5 |
| NCIMB 13365/CIP 78.65) | | |
| Dickeya solani D s0432-1 | DSOIPO2222_RS06570 | tRNA nuclease |
| Serratia proteamaculans (strain 568) | SPRO_RS09200 | phospholipase effector, |
| | | hydrolase |
| Providencia stuartii | S70_RS13555 | triacylglycerol lipase |
| Pseudomonas syringae pv. Tomato | PSPTO_3485 | triacylglycerol lipase |
| (strain DC3000) | | |
| | PSPTO_5438 | TC toxin, toxin |
| Pseudomonas fluorescens F113 | PSF113_RS32820 | Dnase |
| | PSF113_RS60005 | triacylglycerol lipase |
| Pseudomonas fluorescens (strain Pf0-1) | PFL01_RS00775 | triacylglycerol lipase |
| | PFL01_RS10305 | TC toxin |
| Pseudomonas entomophila (strain L48) | PSEEN_RS18405 | TC toxin, toxin |
| | PSEEN_RS25365 | triacylglycerol lipase |
| | PSEEN_RS15865 | triacylglycerol lipase |
| Pseudomonas aeruginosa (strain ATCC | PA3907 | Restriction endonuclease fold |
| 15692/PAO1/1C/PRS 101/LMG | | toxin 5 |
| 12228) | | |
| Pseudomonas mendocina (strain ymp) | PMEN_RS04060 | triacylglycerol lipase |
| Pseudomonas resinovorans NBRC | PCA10_RS00835 | phospholipase effector, |
| 106553 | | hydrolase |
| Ralstonia solanacearum (strain | RS_RS18000 | phospholipase effector, |
| GMI1000) (Pseudomonas solanacearum) | | hydrolase |
| Burkholderia glumae (strain BGR1) | BGLU_RS14230 | Tox-REase-5: Restriction |
| | | endonuclease fold toxin 5 |
| | BGLU_RS18970 | phospholipase effector, |
| | | hydrolase; |
| Burkholderia cenocepacia (strain ATCC | QU43_RS43320 | phospholipase effector, |
| BAA-245/DSM 16553/LMG 16656/ | | hydrolase |
| NCTC 13227/J2315/CF5610) | | |
| (Burkholderia cepacia (strain J2315)) | | |
| | QU43_RS54630 | phospholipase effector, |
| | | hydrolase |
| | QU43_RS43285 | phospholipase effector, |
| | | hydrolase |
| Ralstonia solanacearum FQY_4 | F504_RS17730 | phospholipase effector, |
| | | hydrolase |
| | F504_RS15210 | Tox-REase-5: Restriction |
| | | endonuclease fold toxin 5 |
| Ralstonia solanacearum CFBP2957 | RCFBP_mp30241 | phospholipase effector, |
| | | hydrolase |
| | RCFBP_mp10174 | phospholipase effector, |
| | | hydrolase |
| Variovorax paradoxus (strain EPS) | VARPA_RS28555 | phospholipase effector, |
| | | hydrolase |
| Herbaspirillum seropedicae (strain | HSERO_RS03840 | phospholipase effector, |
| SmR1) | | hydrolase |
| | HSERO_RS04590 | Tox-REase-5: Restriction |
| | | endonuclease fold toxin 5 |
| Chromobacterium violaceum (strain | CV_RS19675 | phospholipase effector, |
| ATCC 12472/DSM 30191/JCM 1249/ | | hydrolase |
| NBRC 12614/NCIMB 9131/NCTC | | |
| 9757) | | |

TABLE 2

| Species | Protein |
|---|---|
| Shewanella woodyi (strain ATCC 51908) | Swoo_2509 |
| Marinobacter aquaeolei (strain ATCC 700491) | Maqu_3717 |
| Photobacterium profundum (Photobacterium sp. (strain SS9)) | PBPRA2069 |
| | PBPRB0619 |
| Vibrio fischeri (strain MJ11) | VFMJ11_A1067 |
| Xanthomonas oryzae pv. oryzae (strain MAFF 311018) | XOO_1614 |
| | XOO_2662 |
| | XOO_1338 |
| | XOO_1344 |
| | XOO_3284 |
| | XOO_3302 |
| | XOO_3312 |
| Proteus mirabilis (strain HI4320) | PMI2993 |
| Enterobacter aerogenes (strain ATCC 13048 | EAE_21965 |
| | EAE_00215 |
| Pectobacterium carotovorum subsp. carotovorum (strain PC1) | PC1_2186 |
| | PC1_3234 |
| Erwinia carotovora subsp. atroseptica (strain SCRI 1043/ATCC BAA-672) | ECA3422 |
| | ECA2106 |
| | ECA3481 |
| | ECA4144 |
| | ECA3421 |
| Photorhabdus asymbiotica subsp. asymbiotica (strain ATCC 43949 | PAU_03046 |
| | PAU_00271 |
| | PAU_03829 |
| | PAU_00713 |
| | PAU_00260 |
| | PAU_04096 |
| | PAU_00510 |
| Dickeya dadantii (strain Ech586) | Dd586_1278 |
| Xenorhabdus bovienii (strain SS-2004) | XBJ1_3597 |
| | XBJ1_1085 |
| | XBJ1_0562 |
| Alkalilimnicola ehrlichei (strain MLHE-1) | Mlg_0041 |
| | Mlg_1143 |
| | Mlg_0840 |
| | Mlg_0648 |
| Hahella chejuensis (strain KCTC 2396) | HCH_03767 |
| | HCH_04401 |
| Marinomonas posidonica (strain CECT 7376/NCIMB 14433/IVIA-Po-181) | Mar181_2499 |
| Chromohalobacter salexigens (strain DSM 3043/ATCC BAA-138/NCIMB | Csal_2252 |
| 13768) | |
| | Csal_0206 |
| | Csal_2276 |
| | Csal_2248 |
| Kangiella koreensis (strain DSM 16069/KCTC 12182/SW-125) | Kkor_1695 |
| Pseudomonas syringae pv. tomato (strain DC3000 | PSPTO_3851 |
| Pseudomonas mendocina (strain ymp) | Pmen_4490 |
| | Pmen_0818 |
| Pseudomonas aeruginosa (strain UCBPP-PA14) | PA14_21470 |
| | PA14_69520 |
| | PA14_13370 |
| Pseudomonas fluorescens (strain Pf0-1) | Pfl01_0632 |
| | Pfl01_0151 |
| | Pfl01_2629 |
| | Pfl01_3047 |
| Acinetobacter sp. (strain ADP1) | ACIAD1790 |
| Ralstonia solanacearum (strain GMI1000) (Pseudomonas solanacearum) | RSp0177 |
| | RSp0755 |
| | RSp0752 |
| Burkholderia cepacia (strain J2315/LMG 16656) | BCAL1364 |
| | BCAM0045 |
| | BCAL1357 |
| Burkholderia ambifaria (strain MC40-6) | BamMC406_6438 |
| | BamMC406_3325 |
| | BamMC406_0412 |
| | BamMC406_1283 |
| Burkholderia thailandensis (strain E264/ATCC 700388/DSM 13276/CIP | BTH_I2102 |
| 106301) | |
| | BTH_II1529 |
| | BTH_I1926 |
| | BTH_II2328 |
| | BTH_I2703 |
| Herbaspirillum seropedicae (strain SmR1) | Hsero_0765 |
| | Hsero_3407 |
| Acidovorax citrulli (strain AAC00-1) (Acidovorax avenae subsp. citrulli) | Aave_0236 |
| Dechloromonas aromatica (strain RCB) | Daro_2167 |
| Chromobacterium violaceum (strain ATCC 12472/DSM 30191/JCM 1249/ | CV_0014 |
| NBRC 12614/NCIMB 9131/NCTC 9757) | |
| | CV_3973 |
| | CV_3988 |
| Sorangium cellulosum (strain So ce56) (Polyangium cellulosum (strain So | sce2706 |
| ce56)) | |
| | sce2707 |
| | sce2474 |
| | sce4668 |
| Geobacter bemidjiensis (strain Bem/ATCC BAA-1014/DSM 16622) | Gbem_3033 |
| Aeromonas hydrophila subsp. hydrophila (strain ATCC 7966/NCIB 9240) | AHA_1121 |
| Klebsiella pneumoniae subsp. pneumoniae HS11286 | KPHS_23060 |
| Escherichia coli O81 (strain ED1a) | ECED1_0244 |
| Photorhabdus luminescens subsp. laumondii (strain TT01) | plu4220 |
| | plu3244 |
| | plu1928 |
| Escherichia coli O44:H18 (strain 042/EAEC) | EC042_0229 |
| Burkholderia pseudomallei (strain K96243) | BPSL2048A |
| | BPSL2039 |
| | BPSL2087 |
| Aggregatibacter aphrophilus (strain NJ8700) | NT05HA_RS09845 |
| Aliivibrio salmonicida (strain LFI1238) | VSAL_RS21910 |
| | VSAL_RS07310 |
| Vibrio fischeri (strain MJ11) | VFMJ11_RS14500 |
| | VFMJ11_RS05175 |
| | VFMJ11_RS13595 |
| Xanthomonas oryzae pv. oryzicola (strain BLS256) | XOC_RS06035 |
| | XOC_RS11220 |
| | XOC_RS06775 |
| Xanthomonas oryzae pv. oryzae (strain KACC10331/KXO85) | XOO_RS22795 |
| | XOO_RS08405 |
| | XOO_RS17140 |
| Alkalilimnicola ehrlichii (strain ATCC BAA-1101/DSM 17681/MLHE-1) | MLG_RS00215 |
| | MLG_RS03345 |
| | MLG_RS04345 |
| Proteus mirabilis (strain HI4320) | PMI_RS14800 |
| Photorhabdus asymbiotica subsp. asymbiotica (strain ATCC 43949/3105-77) | PAU_RS01350 |
| | PAU_RS18980 |
| | PAU_RS06560 |
| | PAU_RS01290 |
| Pectobacterium atrosepticum | ECA_RS10380 |
| Rahnella aquatilis (strain ATCC 33071/DSM 4594/JCM 1683/NBRC 105701/ | RAHAQ2_RS02765 |
| NCIMB 13365/CIP 78.65) | |
| Providencia stuartii | S70_RS06200 |
| | S70_RS09315 |
| Marinomonas posidonica (strain CECT 7376/NCIMB 14433/IVIA-Po-181) | MAR181_RS12715 |
| Hahella chejuensis (strain KCTC 2396 | HCH_RS25155 |
| | HCH_RS16855 |
| | HCH_RS19655 |
| Marinomonas sp. (strain MWYL1) | Mmwyl1_1185 |
| | Mmwyl1_1214 |
| | Mmwyl1_1191 |
| Chromohalobacter salexigens (strain DSM 3043/ATCC BAA-138/NCIMB | CSAL_RS11465 |
| 13768) | |
| | CSAL_RS01035 |
| | CSAL_RS11580 |
| | CSAL_RS11445 |
| Halomonas elongata (strain ATCC 33173/DSM 2581/NBRC 15536/NCIMB | HELO_RS10465 |
| 2198/1H9) | |
| | HELO_RS10360 |
| Kangiella koreensis (strain DSM 16069/KCTC 12182/SW-125) | KKOR_RS08500 |
| Pseudomonas putida H8234 | L483_RS13585 |
| Pseudomonas syringae pv. tomato (strain DC3000) | PSPTO_2536 |
| | PSPTO_3851 |
| Pseudomonas putida (strain KT2440) | PP_3388 |
| | PP_2612 |
| Pseudomonas fluorescens (strain Pf0-1) | PFL01_RS13250 |
| | PFL01_RS15305 |
| | PFL01_RS02300 |
| Pseudomonas entomophila (strain L48) | PSEEN_RS07315 |
| Pseudomonas denitrificans ATCC 13867 | H681_RS03975 |
| Pseudomonas mendocina (strain ymp) | PMEN_RS22630 |
| Ralstonia solanacearum FQY_4 | F504_RS21955 |
| Ralstonia solanacearum CFBP2957 | RCFBP_RS00200 |
| | RCFBP_RS00130 |
| Variovorax paradoxus (strain EPS) | VARPA_RS27590 |
| | VARPA_RS26315 |
| Chromobacterium violaceum (strain ATCC 12472/DSM 30191/JCM 1249/ | CV_RS19765 |
| NBRC 12614/NCIMB 9131/NCTC 9757) | |
| Sorangium cellulosum (strain So ce56) (Polyangium cellulosum (strain So | SCE_RS13865 |
| ce56)) | |
| Xanthomonas oryzae pv. oryzae (strain MAFF 311018) | XOO_1352 |
| Burkholderia pseudomallei (strain K96243) | BPSL1387 |
| Pseudomonas fluorescens (strain Pf0-1) | Pfl01_0456 |
| Sorangium cellulosum (strain So ce56) (Polyangium cellulosum (strain So | SCE_RS23965 |
| ce56)) | |

Experiments also showed that TseC is a T6SS secreted antibacterial effector and the downstream gene tsiC encodes the cognate immunity protein. Other cognate immunity proteins found using the method of the invention are listed in Table 3 below:

TABLE 3
Immunity Protein:

AHA_1122; BGLU_RS14235; BGLU_RS18975;
DSOIPO2222_RS06585; ECA_RS16865; ECA_RS16865; F504_RS15205;
F504_RS17735; HSERO_RS04585; PA3908; PCA10_RS00845; PFL01_RS10300;
PMEN_RS04055; PMI_RS01015; PMI_RS06405; PSEEN_RS18400; PSEEN_RS25355;
PSF113_RS60010; PSPTO_2534; PSPTO_3486; PSPTO_3486; PSPTO_5439, PSPTO_5439;
RAHAQ2_RS22130; RCFBP_mp10175; RCFBP_mp30242; RS_RS18005; S70_RS13545;
SPRO_RS09205; WP_011094985; WP_011148678; WP_011333484; WP_011409743;
WP_011785618; WP_011786021; WP_012325125; WP_012325125; WP_012534013;
WP_012884000; WP_012986793; WP_012988018; WP_013232945; WP_015465491;
WP_015833495; WP_015834617; WP_015841388; WP_041182986; WP_041183642;
WP_042465828; XOO_RS17285; XOO_RS22040 and; YP_108009.

In some embodiments, it was discovered that TseC secretion requires its cognate TEC protein and an associated VgrG protein. Distinct from previous effector-dependent bioinformatic analyses, methods of the present invention use the conserved TEC domain to facilitate the discovery and functional characterization of new T6SS effectors in Gram-negative bacteria.

One aspect of the invention provides a method for identifying a type VI secretion system (“T6SS”) effector in a Gram-negative bacteria, said method comprising:
(a) identifying a conserved domain sequence of T6SS of a Gram-negative bacteria;
(b) searching upstream and downstream of said conserved domain sequence of T6SS of said Gram-negative bacteria;
(c) producing a mutant type of said Gram-negative bacteria by deleting a gene upstream or downstream from said conserved domain sequence of T6SS, wherein said deleted gene is within ten gene sequences from said conserved domain sequence of T6SS;
(d) determining the antibacterial activity of said mutant compared to a wild-type; and
(e) identifying a T6SS effector based on the observed antimicrobial activity of said mutant and said wild-type.

In one embodiment of the invention, a gene that is downstream from the conserved domain sequence of T6SS is deleted in identifying a T6SS effector.

Yet in another embodiment, the deleted gene is within eight, typically within six, and often within five gene sequence from the conserved domain sequence of T6SS.

Still in other embodiments, prior to said step (e), the method of the invention can further include repeating said steps (c) and (d) until a difference in the antibacterial activity between said mutant and said wild-type is observed.

In one particular embodiment, the conserved domain sequence of T6SS comprises VC1417 gene. Within this embodiment, in some instances the conserved domain sequence of T6SS comprises a conserved domain DUF4123.

The method of the invention can further comprise the step of identifying a T6SS effector immunity protein. Such a step generally includes:
(i) producing a second mutant type of said Gram-negative bacteria by deleting a gene upstream or downstream from said conserved domain sequence of T6SS, wherein said deleted gene is within ten gene sequences from said conserved domain sequence of T6SS;
(ii) culturing said second mutant type in the presence of said wild-type; and
(iii) identifying a T6SS effector immunity protein based on the survival of said mutant type in the presence of said wild-type.

Another aspect of the invention provides a method for treating bacterial infection in a subject comprising administering to a subject in need of bacterial infection treatment a therapeutically effective amount of a composition comprising a type VI secretion system (T6SS) effector discovered using a method of the invention disclosed herein.

Exemplary T6SS effectors that have been found by method of the invention and can be used to treat a subject in need of antibacterial treatment include a protein listed in Tables 1 and 2.

Still another aspect of the invention provides a method for treating a Gram-negative bacteria infection in a subject, said method comprising administering to a subject in need of Gram-negative bacterial infection treatment a therapeutically effective amount of a composition comprising a type VI secretion system (T6SS) effector immunity protein discovered using a method of the invention disclosed herein.

Exemplary T6SS effector immunity proteins that have been found by the method of the invention and can be used to treat a subject suffering from Gram-negative bacteria include a protein that is encoded by a gene listed in Table 3.

### BRIEF DESCRIPTION OF THE DRAWINGS

FIG. 1: Panel A is a schematic illustration of operon structure of the VC1415-VC1421 region. Panel (B) is immunoblotting analysis of TseL::3V5 secretion as detected in the cytosolic (Cell) and supernatant (Sec) fractions. Panel (C) is a graph showing VC1417 is required for delivery of TseL. Panel (D) is a immunoblotting showing VC1417 is not secreted by T6SS. Panel (E) is a graph showing effects of VgrG proteins on TseL delivery. Panel (F) is a result showing bacterial two-hybrid analysis of VC1417 interaction with VgrG1 and TseL. Panel (G) is shows co-immunoprecipitation of VC1417 with VgrG1 and TseL.

FIG. 2 is schematic illustration of known and predicted DUF4123 associated effectors.

FIG. 3: Panel (A) shows T6SS-dependent killing of E. coli by SSU. Panel (B) shows T6SS-dependent secretion of TseC. Panel (C) is schematic illustration of operon structure of SSU tseC-tsiC. Panel (D) shows expression of the SSU TseC colicin domain (768-891) is toxic in E. coli. Panel (E) shows TsiC is the cognate immunity protein to TseC. Panel (F) shows ORF2403 and VgrG1 (ORF2404) are required for killing the tseC tsiC mutant but not E. coli. Panel (G) shows bacterial two-hybrid assay of the interaction of ORF2403 with VgrG1(ORF2404) and TseC. Panel (H) shows co-immunoprecipitation analysis of the interaction of ORF2403 with VgrG1(ORF2404) and TseC.

FIG. 4 is a test result showing T6SS-mediated killing of E. coli by V. cholerae mutants.

FIG. 5 is a test result showing T6SS-mediated killing of E. coli by SSU tseC mutant.

FIG. 6 is a schematic illustration showing DUF4123 protein domain has several highly conserved residues.

FIG. 7(A) is a schematic illustration showing operon structures of SSU928.

FIG. 7(b) is a graph of test results showing complementation with immunity genes confers protection.

FIG. 8A is a schematic illustration showing operon structure of the PA3907 gene cluster. A deletion mutant lacking both PA3907 and PA3908 was constructed.

FIG. 8B is a schematic illustration showing PA3907 possess a conserved toxic Tox-REase-5 functional domain.

FIG. 8C is a graph showing the deletion mutant of PA3907 and PA3908 was efficiently killed by wild type Pseudomonas aeruginosa but not by the H2-T6SS cluster mutant T6S-2.

FIG. 9: Panel (A) is a schematic illustration of operon structure of the effector VC661 and it immunity gene VC661i. Panel (B) shows expression of VC661 in the periplasm of E. coli results in extensive cell lysis, indicating severe damage to the cell wall

### DETAILED DESCRIPTION OF THE INVENTION

Some aspects of the invention provide a method for identifying T6SS effectors and/or T6SS effector immunity protein in a Gram-negative bacteria. T6SS is a specialized protein delivery system that many Gram-negative bacteria use to kill eukaryotic and prokaryotic competitors by translocating toxic protein molecules (i.e., T6SS effectors) to target cells. Identification of effectors is required for understanding the pivotal role that the T6SS plays in dictating interbacterial and bacterial-host dynamics. In one particular aspect, the present invention provides a new approach to identifying T6SS effectors. As described herein, secretion of effectors requires interaction with a set of cognate effector-binding chaperone proteins that are also disclosed herein. These and other discoveries disclosed herein by the present inventors provides important insights for understanding the mechanism of T6SS effector delivery as well as identifying T6SS effector immunity protein that can be used for treatment in a subject infected with Gram-negative bacteria.

Protein secretion systems play a pivotal role in bacterial interspecies interaction and virulence (1, 2). Of the known secretion systems in Gram-negative bacteria, the type VI secretion system (T6SS) enables bacteria to compete with both eukaryotic and prokaryotic species through delivery of toxic effectors (2-4). The T6SS is a multicomponent nanomachine analogous to the contractile bacteriophage tail (5). First characterized in Vibrio cholerae (6) and Pseudomonas aeruginosa (7), the T6SS has now been identified in ˜25% Gram-negative bacteria including many important pathogens (2, 8), and implicated as a critical factor in niche competition (9-11).

T6SS structure is composed of an Hcp inner tube, a VipAB outer sheath that wraps around the Hcp tube, a tip complex consisting of VgrG and PAAR proteins, and a membrane-bound baseplate (2, 4, 12). Sheath contraction drives the inner Hcp tube and the tip proteins, VgrG and PAAR, outward into the environment and neighboring cells (13, 14). The contracted sheath is then dissembled by an ATPase ClpV and recycled for another T6SS assembly and contraction event (12, 15, 16). Two essential T6SS baseplate components VasF and VasK are homologous to the DotU and IcmF proteins of the type IV secretion system (T4SS) in Legionella pneumophila (17).

Bacteria often possess multiple copies of VgrG and PAAR genes that form the tip of T6SS, and deletion of VgrG and PAAR genes abolishes T6SS secretion (14). Some VgrG and PAAR proteins carry functional extension domains and thus act as secreted T6SS effectors, as exemplified by the VgrG1 actin crosslinking domain (6), VgrG3 lysozyme domain in V. cholerae (18, 19), and the nuclease domain of the PAAR protein RhsA in Dickeya dadantii (20). Known T6SS effectors can target a number of essential cellular components including the actin and membrane of eukaryotic cells (18, 21, 22) and the cell wall, membrane, and DNA of bacterial cells (3, 18-20, 23, 24). Each antibacterial effector coexists with an antagonistic immunity protein that confers protection during T6SS-mediated attacks between sister cells (3, 18, 24). Interestingly, T6SS-mediated lethal attacks induce the generation of reactive oxygen species in the prey cells (25), similar to cells treated with antibiotics (26, 27).

For non-VgrG/PAAR related effectors, their translocation requires either binding to the inner tube Hcp proteins as chaperones or binding to the tip VgrG proteins (2, 14, 28). T6SS-dependent effectors can be experimentally identified by comparing the secretomes of wild type and T6SS mutants (3, 29-31) and by screening for T6SS-encoded immunity proteins (18). Because known effectors lack a common secretion signal, bioinformatic identification of T6SS effectors is challenging. A heuristic approach based on the physical properties of effectors has been used to identify a superfamily of peptidoglycan-degrading effectors in bacteria (32). A recent study identified a common N-terminal motif in a number of T6SS effectors (31). However, this motif does not exist in the T6SS effector TseL in V. cholerae (18).

One aspect of the invention is based on discovery by the present inventors of VC1417 gene that encodes a protein with a highly conserved domain, namely DUF4123. VC1417 gene is located upstream of tseL. As shown herein, VC1417 is required for TseL delivery and interacts with VgrG1 (VC1416) and TseL. Because of the genetic linkage of VC1417 and TseL and its importance for TseL secretion, it is believed that genes encoding the conserved DUF4123 domain proteins are generally located upstream of genes encoding putative T6SS effectors. Using the conserved domain sequence, the present inventors bioinformatically predicted a large family of effector proteins with diverse functions in Gram-negative bacteria. The method of the invention was used for identification and characterization of a new secreted effector TseC and its antagonistic immunity protein TsiC in A. hydrophila SSU. Results from the method of the invention demonstrate a new effective approach to identify T6SS effectors with highly divergent sequences.

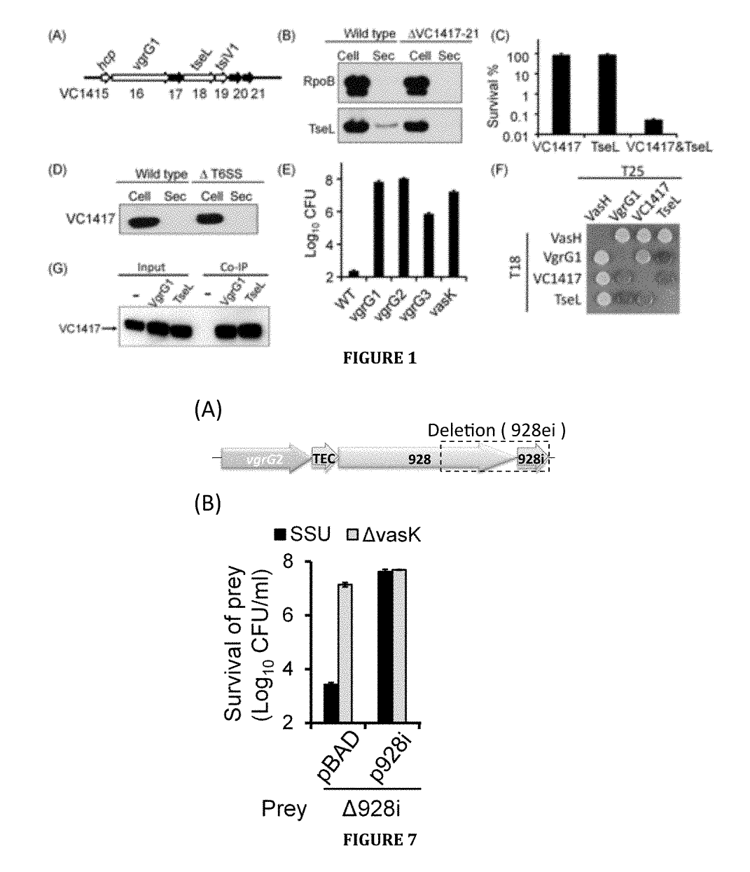

TseL (VC1418) is located in the V. cholerae hcp1 operon consisting of 7 genes (FIG. 1A), three of which (VC1417, VC1420, and VC1421) encode proteins with unknown functions (indicated in black on FIG. 1A). The present inventors investigated whether any of these three genes were required for TseL secretion. By comparing wild type and a mutant lacking VC1417 to VC1421 (33), it was found that TseL cannot be secreted in the mutant (FIG. 1B), indicating at least one of the three genes, VC1417, VC1420, and VC1421, is required for TseL secretion. It was reasoned that if complementation of a gene can restore TseL secretion in the VC1417-21 mutant that lacks the immunity gene VC1419, it would be highly toxic when co-expressed with TseL due to sister cell to sister cell delivery of TseL. Indeed, it was found that co-expression of VC1417 and TseL reduced cell survival by 10³-fold while expressing VC1417 or TseL alone had little effect on cell viability (FIG. 1C), indicating that VC1417 is required for TseL secretion. Interestingly, VC1417 itself is not a secreted substrate of T6SS (FIG. 1D). Expression of VC1420 or VC1421 did not restore TseL secretion.

It was then tested whether the vgrG1 gene, upstream of VC1417, is involved in TseL secretion in V. cholerae. Using the VC1417-21 mutant as a prey, it was found that wild type V. cholerae killed the prey efficiently while the vgrG2 mutant could not kill (FIG. 1E). This is consistent with previous reports that VgrG2 is essential for T6SS secretion (18, 33). Interestingly, while the vgrG3 mutant exhibited impaired killing, killing was completely abolished in the vgrG1 mutant (FIG. 1E). As a control, the vgrG1 mutant efficiently killed E. coli as a prey (FIG. 4), consistent with a previous report (33). These results indicate VgrG1 is not essential for T6SS activity but is required for delivery of TseL. The present inventors have previously found that VgrG3 interacts with TseL in V. cholerae but not in E. coli (18). Because VgrG proteins likely form a heterotrimer in V. cholerae (21, 33), the present findings suggest that the previously reported interaction between VgrG3 and TseL is likely through VgrG1.

The requirement of VC1417 and VgrG1 for TseL delivery indicates that these proteins interact with one another. To test this, a bacterial two-hybrid assay based on the functional complementation of the two T18 and T25 fragments of Bordetella pertussis adenylate cyclase were used (34). Protein interaction functionally reconstitutes the activity of adenylate cyclase that subsequently results in a LacZ⁺ phenotype on LB supplemented with X-gal. Because VgrG1 carries a large C-terminal actin-crosslinking domain that can be swapped by beta-lactamase without affecting secretion (35), it was reasoned that the C-terminal extension domain is not required for delivery and thus only expressed the highly conserved N-terminal sequence (1-638aa) of VgrG1 for testing protein-protein interaction. It was found that LacZ⁺ phenotypes when VC1417 was co-expressed with TseL or VgrG1, indicating direct interaction (FIG. 1F). In contrast, the negative control VasH, a DNA-binding sigma54-dependent regulator (36-38), exhibited LacT when co-expressed with the other proteins tested. Using co-immunoprecipitation assays, the present inventors confirmed the interaction of VC1417 with VgrG1 and TseL (FIG. 1G).

By searching the protein sequence of VC1417 in the Pfam protein database (39), it was found that VC1417 carries a conserved domain DUF4123. Moreover, DUF4123 was found in 818 protein sequences in 344 bacterial species, 342 of which belong to Proteobacteria including Gammaproteobacteria (69%) and Betaproteobacteria (26%). Although DUF4123 is the only domain in the majority of these proteins, a few proteins carry an additional FHA domain (forkhead-associated) that is often involved in regulatory functions through phosphorylation. Interestingly, the Fha1 protein in P. aeruginosa is required for activating the T6SS cluster 1 (40). In addition, over 90% of DUF4123-encoding bacterial genomes also carry hallmark T6SS proteins, VipA (the outer sheath) and Hcp (the inner tube), indicating a strong association between the presence of DUF4123 and T6SS genes.

The genome of V. cholerae encodes another DUF4123 domain protein, VasW, which is known to be required for secretion of its downstream effector VasX (23). Because the DUF4123 domain proteins are widely distributed in Gram-negative bacteria, it was reasoned that this conserved domain could be used as a signal to find highly divergent T6SS effectors. Two previously characterized effectors in V. cholerae, TseL and VasX, share little sequence similarity but both have the DUF4123 domain containing genes upstream, validating this method as a potential strategy for T6SS effector identification.

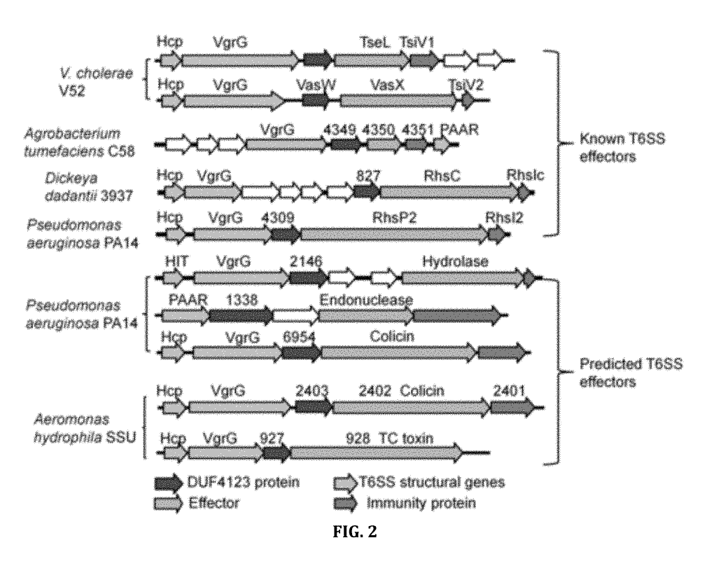

DUF4123 and downstream genes were first examined in a number of known T6SS-active bacteria, including P. aeruginosa (41), Agrobacterium tumefaciens (10), Dickeya dadantii (20), and Aeromonas hydrophila (42). Genes encoding the DUF4123 domain were found upstream of genes encoding known T6SS effectors. Notably, DUF4123 genes were also often located together with at least of one of the genes encoding T6SS secreted proteins, Hcp, VgrG, or PAAR (FIG. 2).

Whether the DUF4123 domain can predict unknown T6SS effectors were tested. P. aeruginosa PA14 carries four DUF4123 proteins, one of which is located upstream of a known T6SS effector RhsP2 (41). The other three DUF4123 genes are located immediately downstream of genes encoding VgrG or PAAR proteins (FIG. 2). Using a structural prediction program HHpred (43), it was found that the three genes downstream each of the DUF4123 genes encode a hydrolase, a endonuclease, and a colicin, respectively, indicating these are T6SS effectors. There are two DUF4123 domain proteins in A. hydrophila SSU (FIG. 2). Sequence analyses using HHpred (43) and Phyre2 (44, 45) show that the ORF2402 carries a putative colicin domain (HHpred probability 21% and Phyre2 confidence 78%) and the ORF0928 carries a TC toxin domain (HHpred probability 100% and Phyre2 confidence 100%). The TC toxin complexes are important virulence factors in many bacterial pathogens, including Photorhabdus luminescens and Yersinia pestis, which target insects and mammalian cells (46, 47). Thus, these were named ORF2402 TseC for its colicin domain and ORF0928 TseI for its potential insecticidal activity.

Analysis was expanded to 43 representative bacterial species with completely annotated genomes that encode the DUF4123 proteins. For genes immediately upstream of the 133 DUF4123 genes analyzed, 70% possess an upstream vgrG and 7.5% an upstream PAAR (Table A). For DUF4123 downstream genes, while the majority encode unknown functions, genes with known/predicted functions encode putative TC-toxin, lipase, nuclease, and hydrolase.

TABLE A

| | | | | | | Predicted |
|---|---|---|---|---|---|---|
| | DUF4123 | | | Predicted | | Function of |
| | protein | DUF4123 | Upstream | function of | Downstream | downstream |
| Species | (UnipProt) | gene | gene | upstream gene | gene | gene |
| Agrobacterium | Q7CUQ0 | Atu4349 | Atu4348 | VgrG | Atu4350 | Rnase toxin, |
| tumefaciens | | | | | | toxin_43 |
| C58 | | | | | | |
| Shewanella | B1KGR7 | Swoo_2509 | Swoo_2510 | unknown | Swoo_2508 | TC toxin, toxin |
| woodyi (strain | | | | function | | |
| ATCC 51908) | | | | | | |
| | B1KGR7 | Swoo_2510 | Swoo_2511 | ANKYRIN REPEAT | Swoo_2509 | unknown |
| | | | | AND PROTEIN | | function |
| | | | | KINASE DOMAIN- | | |
| | | | | CONTAINING | | |
| | | | | PROTEIN | | |
| Marinobacter | A1U3S0 | Maqu_2578 | Maqu_2579 | VgrG | Maqu_2577 | TC toxin, toxin |
| aquaeolei | | | | | | |
| (strain ATCC | | | | | | |
| 700491) | | | | | | |
| | A1U3S0 | Maqu_2564 | Maqu_2565 | VgrG | Maqu_2563 | TC toxin, toxin |
| | A1U3S0 | Maqu_2571 | Maqu_2572 | VgrG | Maqu_2570 | TC toxin, toxin |
| | A1U718 | Maqu_3718 | Maqu_3719 | VgrG | Maqu_3717 | unknown |
| | | | | | | function |
| | A1U2L0 | Maqu_2150 | Maqu_2151 | VgrG | Maqu_2149 | Cell wall |
| | | | | | | hydrolyses, |
| | | | | | | Autolysin |
| Photobacterium | Q6LQF4 | PBPRA2070 | PBPRA2072 | unknown | PBPRA2069 | unknown |
| profundum | | | | function | | function |
| (Photobacterium | | | | | | |
| sp. (strain | | | | | | |
| SS9)) | | | | | | |
| | Q6LJN9 | PBPRB0618 | PBPRB0617 | nudix protein | PBPRB0619 | unknown |
| | | | | | | function |
| Vibrio fischeri | B5FEE7 | VFMJ11_1493 | VFMJ11_1494 | VgrG | VFMJ11_1492 | lipoprotein |
| (strain MJ11) | | | | | | NlpD; |
| | | | | | | Provisional |
| | B5EV98 | VFMJ11_A1068 | VFMJ11_A1069 | VgrG | VFMJ11_A1067 | unknown |
| | | | | | | function |
| | B5FDW6 | VFMJ11_1310 | VFMJ11_1309 | VgrG | VFMJ11_1311 | Nucleoporin, |
| | | | | | | Vacuolar |
| | | | | | | transporter |
| | | | | | | chaperone |
| Vibrio cholerae | Q9KNE6 | VC_A0019 | VC_A0018 | VgrG | VC_A0020 | Colicin |
| O1 El Tor | | | | | | |
| N16961 | | | | | | |
| | Q9KS44 | VC1417 | VC1416 | VgrG | VC1418 | Lipase |
| Aeromonas | Q6TP06 | AHA_1120 | AHA_1119 | VgrG | AHA_1121 | Hydrolase |
| hydrophila | | | | | | |
| ATCC7966 | | | | | | |
| Xanthomonas | Q2P5S1 | XOO_1351 | XOO_1350 | VgrG | XOO_1352 | unknown |
| oryzae pv. | | | | | | function |
| oryzae (strain | | | | | | |
| MAFF 311018) | | | | | | |
| | Q2P507 | XOO_1615 | XOO_1616 | VgrG | XOO_1614 | unknown |
| | | | | | | function |
| | Q2P209 | XOO_2663 | XOO_2664 | VgrG | XOO_2662 | unknown |
| | | | | | | function |
| | Q2P5T5 | XOO_1337 | XOO_1336 | VgrG | XOO_1338 | unknown |
| | | | | | | function |
| | Q2NXL8 | XOO_4204 | XOO_4205 | PAAR | XOO_4203 | Arsenate |
| | | | | | | reductase |
| | Q2P5S9 | XOO_1343 | XOO_1342 | VgrG | XOO_1344 | unknown |
| | | | | | | function |
| | Q2P087 | XOO_3285 | XOO_3286 | VgrG | XOO_3284 | unknown |
| | | | | | | function |
| | Q2P069 | XOO_3303 | XOO_3304 | VgrG | XOO_3302 | unknown |
| | | | | | | function |
| | Q2P059 | XOO_3313 | XOO_3314 | VgrG | XOO_3312 | unknown |
| | | | | | | function |
| | Q2NX71 | XOO_4351 | XOO_4352 | PAAR | XOO_4350 | Restriction |
| | | | | | | endonuclease |
| | | | | | | fold toxin 5 |
| Proteus | B4F003 | PMI2992 | PMI2991 | VgrG | PMI2993 | unknown |
| mirabilis | | | | | | function |
| (strain HI4320) | | | | | | |
| | B4EUE5 | PMI0209 | PMI0208 | VgrG | PMI0210 | Triacylglycerol |
| | | | | | | lipase |
| | B4EW21 | PMI1330 | PMI1331 | VgrG | PMI1329 | triacylglycerol |
| | | | | | | lipase |
| Enterobacter | G0DZR5 | EAE_21970 | EAE_21975 | Condensing | EAE_21965 | unknown |
| aerogenes | | | | enzymes | | function |
| (strain ATCC | | | | | | |
| 13048 | | | | | | |
| | G0E9C6 | EAE_00220 | EAE_00225 | cupin_WbuC | EAE_00215 | unknown |
| | | | | cupin fold | | function |
| | | | | metalloprotein, | | |
| | | | | WbuC family | | |
| Pectobacterium | C6DCS9 | PC1_3234 | PC1_3235 | unknown | PC1_3233 | TC toxin, toxin |
| carotovorum | | | | function | | |
| subsp. | | | | | | |
| carotovorum | | | | | | |
| (strain PC1) | | | | | | |
| | C6DI53 | PC1_2187 | PC1_2188 | VgrG | PC1_2186 | unknown |
| | | | | | | function |
| | C6DCT0 | PC1_3235 | PC1_3236 | ankyrin-like | PC1_3234 | unknown |
| | | | | protein; | | function |
| | | | | Provisional | | |
| Erwinia | Q6D1M2 | ECA3423 | ECA3424 | ankyrin-like | ECA3422 | unknown |
| carotovora | | | | protein; | | function |
| subsp. | | | | Provisional | | |
| atroseptica | | | | | | |
| (strain SCRI | | | | | | |
| 1043/ATCC | | | | | | |
| BAA-672) | | | | | | |
| | I1SBC5 | ECA2105 | ECA2104 | VgrG | ECA2106 | unknown |
| | | | | | | function |
| | Q6D1G4 | ECA3482 | ECA3483 | unknown | ECA3481 | unknown |
| | | | | function | | function |
| | Q6CZK8 | ECA4143 | ECA4142 | VgrG | ECA4144 | unknown |
| | | | | | | function |
| | Q6D1M3 | ECA3422 | ECA3423 | unknown | ECA3421 | unknown |
| | | | | function | | function |
| Photorhabdus | B6VMZ2 | PAU_03047 | PAU_03048 | VgrG | PAU_03046 | unknown |
| asymbiotica | | | | | | function |
| subsp. | | | | | | |
| asymbiotica | | | | | | |
| (strain ATCC | | | | | | |
| 43949 | | | | | | |
| | B6VND9 | orf42 | PAU_02485 | VgrG | pdl1 | triacylglycerol |
| | | | | | PAU_02483 | lipase |
| | B6VM72 | PAU_00272 | PAU_00273 | VgrG | PAU_00271 | unknown |
| | | | | | | function |
| | C7BNW1 | PAU_02638 | PAU_02639 | VgrG | PAU_02637 | PAAR |
| | C7BMN9 | PAU_03830 | PAU_03831 | VgrG | PAU_03829 | unknown |
| | | | | | | function |
| | B6VL31 | orf42 | PAU_02446 | VgrG | zwf | GLUCOSE-6- |
| | | PAU_02445 | | | PAU_02443 | PHOSPHATE 1- |
| | | | | | | DEHYDROGENASE |
| | | | | | | (G6PD) |
| | C7BRN8 | PAU_01347 | PAU_01346 | unknown | PAU_01348 | Transposase |
| | | | | function | | protein |
| | B6VMP8 | PAU_00714 | PAU_00715 | sugar binding | PAU_00713 | unknown |
| | | | | protei, | | function |
| | | | | automated | | |
| | | | | matches {Mouse | | |
| | | | | (Mus musculus) | | |
| | | | | [TaxId: 10090]} | | |
| | B6VM61 | PAU_00261 | PAU_00262 | VgrG | PAU_00260 | unknown |
| | | | | | | function |
| | B6VLN8 | PAU_04095 | PAU_04094 | VgrG | PAU_04096 | unknown |
| | | | | | | function |
| | C7BJT7 | PAU_00511 | PAU_00512 | VgrG | PAU_00510 | unknown |
| | | | | | | function |
| Dickeya | D2BVK8 | Dd586_1278 | Dd586_1277 | unknown | Dd586_1279 | TC toxin, toxin |
| dadantii (strain | | | | function | | |
| Ech586) | | | | | | |
| | D2BVK7 | Dd586_1277 | Dd586_1276 | ankyrin-like | Dd586_1278 | unknown |
| | | | | protein; | | function |
| | | | | Provisional | | |
| Xenorhabdus | D3V7F8 | XBJ1_2646 | XBJ1_2645 | VgrG | XBJ1_2647 | hydroxyisourate |
| bovienii (strain | | | | | | hydrolase, |
| SS-2004) | | | | | | Transthyretin |
| | D3UYF3 | XBJ1_0180 | XBJ1_0181 | VgrG | XBJ1_0179 | triacylglycerol |
| | | | | | | lipase |
| | D3V4Y7 | XBJ1_3598 | ycdH | hpaC 4- | XBJ1_3597 | unknown |
| | | | XBJ1_3599 | hydroxyphenylacetate | | function |
| | | | | 3- | | |
| | | | | monooxygenase | | |
| | | | | reductase | | |
| | | | | subunit; | | |
| | | | | Provisional | | |
| | D3UXB5 | XBJ1_1086 | XBJ1_1087 | VgrG | XBJ1_1085 | unknown |
| | | | | | | function |
| | D3UZC3 | XBJ1_0561 | XBJ1_0560 | VgrG | XBJ1_0562 | unknown |
| | | | | | | function |
| | D3V077 | XBJ1_1502 | XBJ1_1501 | VgrG | XBJ1_1503 | Triacylglycerol |
| | | | | | | lipase |
| Alkalilimnicola ehrlichei | Q0ACN7 | Mlg_0042 | Mlg_0043 | VgrG | Mlg_0041 | unknown |
| (strain MLHE- | | | | | | function |
| 1) | | | | | | |
| | Q0A9J4 | Mlg_1144 | Mlg_1145 | VgrG | Mlg_1143 | unknown |
| | | | | | | function |
| | Q0AAE4 | Mlg_0839 | Mlg_0838 | VgrG | Mlg_0840 | unknown |
| | | | | | | function |
| | Q0QQY4 | Mlg_0649 | Mlg_0650 | VgrG | Mlg_0648 | unknown |
| | | | | | | function |
| Hahella | Q2SAR1 | HCH_05605 | HCH_05604 | unknown | HCH_05606 | ThuA Trehalose |
| chejuensis | | | | function | | utilisation |
| (strain KCTC | | | | | | |
| 2396) | | | | | | |
| | Q2SFS4 | HCH_03768 | HCH_03769 | protein secret | HCH_03767 | unknown |
| | | | | protein transport | | function |
| | Q2SE17 | HCH_04402 | HCH_04403 | Alkylsulfatase | HCH_04401 | unknown |
| | | | | SdsA1, SDS- | | function |
| | | | | hydrolase, | | |
| | | | | lactamase, | | |
| | | | | hydrolase | | |
| Marinomonas | F6CWW9 | Mar181_2498 | Mar181_2497 | VgrG | Mar181_2499 | unknown |
| posidonica | | | | | | function |
| (strain CECT | | | | | | |
| 7376/NCIMB | | | | | | |
| 14433/IVIA- | | | | | | |
| Po-181) | | | | | | |
| Chromohalobacter | Q1QVA5 | Csal_2253 | Csal_2254 | VgrG | Csal_2252 | unknown |
| salexigens | | | | | | function |
| (strain DSM | | | | | | |
| 3043/ATCC | | | | | | |
| BAA-138/ | | | | | | |
| NCIMB 13768) | | | | | | |
| | Q1R139 | Csal_0205 | Csal_0204 | VgrG | Csal_0206 | unknown |
| | | | | | | function |
| | Q1QV83 | Csal_2275 | Csal_2274 | VgrG | Csal_2276 | unknown |
| | | | | | | function |
| | Q1QVA8 | Csal_2249 | Csal_2250 | unknown | Csal_2248 | unknown |
| | | | | function | | function |
| Kangiella | C7RCW6 | Kkor_1696 | Kkor_1697 | GAMMA | Kkor_1695 | unknown |
| koreensis | | | | GLUTAMYL | | function |
| (strain DSM | | | | TRANSPEPTIDASES | | |
| 16069/KCTC | | | | | | |
| 12182/SW- | | | | | | |
| 125) | | | | | | |
| Pseudomonas | Q87ZE6 | PSPTO_3483 | PSPTO_3482 | VgrG | PSPTO_3484 | lipoprotein |
| syringae pv. | | | | | | |
| tomato (strain | | | | | | |
| DC3000 | | | | | | |
| | Q87U71 | PSPTO_5437 | PSPTO_5436 | VgrG | PSPTO_5438 | TC toxin, toxin |
| | Q882T5 | PSPTO_2537 | PSPTO_2538 | VgrG | PSPTO_2536 | TilS-like protein |
| | Q87YF0 | PSPTO_3850 | PSPTO_3849 | VgrG | PSPTO_3851 | unknown |
| | | | | | | function |
| Pseudomonas | A4Y0X0 | Pmen_4489 | Pmen_4488 | VgrG | Pmen_4490 | unknown |
| mendocina | | | | | | function |
| (strain ymp) | | | | | | |
| | A4XQH1 | Pmen_0819 | Pmen_0820 | VgrG | Pmen_0818 | unknown |
| | | | | | | function |
| Pseudomonas | Q02QC6 | PA14_21460 | PA14_21450 | VgrG | PA14_21470 | unknown |
| aeruginosa | | | | | | function |
| (strain UCBPP- | | | | | | |
| PA14) | | | | | | |
| | Q02KE3 | PA14_43090 | PA14_43080 | VgrG | PA14_43100 | TC toxin, toxin |
| | Q02E97 | PA14_69540 | PA14_69550 | VgrG | PA14_69520 | unknown |
| | | | | | | function |
| | Q02S71 | PA14_13380 | PA14_13390 | PAAR | PA14_13370 | unknown |
| | | | | | | function |
| Pseudomonas | Q3KIN3 | Pfl01_0629 | Pfl01_0628 | VgrG | Pfl01_0632 | unknown |
| fluorescens | | | | | | function |
| (strain Pf0-1) | | | | | | |
| | Q3KK10 | Pfl01_0152 | Pfl01_0153 | VgrG | Pfl01_0151 | unknown |
| | | | | | | function |
| | Q3KCY4 | Pfl01_2630 | Pfl01_2631 | VgrG | Pfl01_2629 | unknown |
| | | | | | | function |
| | Q3KEM0 | Pfl01_2043 | Pfl01_2044 | VgrG | Pfl01_2042 | TC toxin, toxin |
| | Q3KBS1 | Pfl01_3045 | Pfl01_3044 | FimD P pilus | Pfl01_3047 | unknown |
| | | | | assembly protein | | function |
| | Q3KJ57 | Pfl01_0455 | Pfl01_0454 | VgrG | Pfl01_0456 | HisKA_3 |
| | | | | | | Histidine kinase |
| Acinetobacter | Q6FBD7 | ACIAD1789 | ACIAD1788 | VgrG | ACIAD1790 | unknown |
| sp. (strain | | | | | | function |
| ADP1) | | | | | | |
| Ralstonia | Q8XTD7 | RSp0176 | RSp0175 | VgrG | RSp0177 | unknown |
| solanacearum | | | | | | function |
| (strain | | | | | | |
| GMI1000) | | | | | | |
| (Pseudomonas | | | | | | |
| solanacearum) | | | | | | |
| | Q8XUP2 | RSc3144 | tlSRso9 | DDE_Tnp_1_4 | RSc3143 | DeoR family |
| | | | RSc3145 | Transposase DDE | | transcriptional |
| | | | | domain group 1 | | regulator |
| | Q8XRS7 | RSp0754 | RSp0753 | unknown | RSp0755 | unknown |
| | | | | function | | function |
| | Q8XRT0 | RSp0751 | RSp0750 | VgrG | RSp0752 | unknown |
| | | | | | | function |
| Burkholderia | B4E683 | BCAL1363 | BCAL1362 | VgrG | BCAL1364 | unknown |
| cepacia (strain | | | | | | function |
| J2315/LMG | | | | | | |
| 16656) | | | | | | |
| | B4EG30 | BCAM0044 | BCAM0043 | VgrG | BCAM0045 | unknown |
| | | | | | | function |
| | B4E677 | BCAL1356 | BCAL1355 | VgrG | BCAL1357 | unknown |
| | | | | | | function |
| Burkholderia | B1Z554 | BamMC406_6439 | BamMC406_6440 | VgrG | BamMC406_6438 | unknown |
| ambifaria | | | | | | function |
| (strain MC40- | | | | | | |
| 6) | | | | | | |
| | B1YYT5 | BamMC406_3326 | BamMC406_3327 | VgrG | BamMC406_3325 | unknown |
| | | | | | | function |
| | B1YSA6 | BamMC406_0411 | BamMC406_0410 | VgrG | BamMC406_0412 | unknown |
| | | | | | | function |
| | B1YNF5 | BamMC406_1282 | BamMC406_1281 | VgrG | BamMC406_1283 | unknown |
| | | | | | | function |
| Burkholderia | Q2SWS3 | BTH_I2103 | BTH_I2104 | PAAR | BTH_I2102 | unknown |
| thailandensis | | | | | | function |
| (strain E264/ | | | | | | |
| ATCC 700388/ | | | | | | |
| DSM 13276/ | | | | | | |
| CIP 106301) | | | | | | |
| | Q2T524 | BTH_II1530 | BTH_II1531 | VgrG | BTH_II1529 | unknown |
| | | | | | | function |
| | Q2T6R8 | BTH_II0934 | BTH_II0933 | VgrG | BTH_II0935 | PHP DOMAIN |
| | | | | | | PROTEIN. |
| | Q2SX92 | BTH_I1927 | BTH_I1928 | PAAR | BTH_I1926 | unknown |
| | | | | | | function |
| | Q2T2T3 | BTH_II2329 | BTH_II2330 | PAAR | BTH_II2328 | unknown |
| | | | | | | function |
| | Q2SV30 | BTH_I2704 | BTH_I2705 | VgrG | BTH_I2703 | unknown |
| | | | | | | function |
| Herbaspirillum | D8J097 | Hsero_0917 | Hsero_0918 | PAAR | Hsero_0916 | putative toxin- |
| seropedicae | | | | | | antitoxin system |
| (strain SmR1) | | | | | | |
| | D8IZG3 | Hsero_0764 | Hsero_0763 | VgrG | Hsero_0765 | unknown |
| | | | | | | function |
| | D8IPJ0 | Hsero_3406 | Hsero_3405 | VgrG | Hsero_3407 | unknown |
| | | | | | | function |
| Acidovorax | A1TIQ6 | Aave_0237 | Aave_0238 | unknown | Aave_0236 | unknown |
| citrulli (strain | | | | function | | function |
| AAC00-1) | | | | | | |
| (Acidovorax | | | | | | |
| avenae subsp. | | | | | | |
| citrulli) | | | | | | |
| Dechloromonas | Qf7E24 | Daro_2168 | Daro_2169 | VgrG | Daro_2167 | unknown |
| aromatica | | | | | | function |
| (strain RCB) | | | | | | |
| Chromobacterium | Q7P246 | CV_0015 | CV_0016 | VgrG | CV_0014 | unknown |
| violaceum | | | | | | function |
| (strain ATCC | | | | | | |
| 12472/DSM | | | | | | |
| 30191/JCM | | | | | | |
| 1249/NBRC | | | | | | |
| 12614/NCIMB | | | | | | |
| 9131/NCTC | | | | | | |
| 9757) | | | | | | |
| | Q7NR11 | CV_3974 | CV_3975 | VgrG | CV_3973 | unknown |
| | | | | | | function |
| | Q7NQZ8 | CV_3987 | CV_3986 | VgrG | CV_3988 | unknown |
| | | | | | | function |
| Sorangium | A9GAG5 | sce2703 | sce2702 | PAAR | sce2706 | unknown |
| cellulosum | | | | | | function |
| (strain Soce56) | | | | | | |
| (Polyangium | | | | | | |
| cellulosum | | | | | | |
| (strain Soce56)) | | | | | | |
| | A9GAH2 | sce2706 | sce2703 | unknown | sce2707 | unknown |
| | | | | function | | function |
| | A9G604 | sce2478 | wzx2 sce2479 | colanic acid | sce2474 | unknown |
| | | | | exporter; | | function |
| | | | | Provisional | | |
| | A9FDY8 | sce4669 | sce4670 | PAAR | sce4668 | unknown |
| | | | | | | function |
| Myxococcus | Q1D4S9 | MXAN_4169 | MXAN_4168 | unknown | MXAN_4178 | ATP-DEPENDENT |
| xanthus (strain | | | | function | | CLP PROTEASE |
| DK 1622) | | | | | | |
| Geobacter | B5E867 | Gbem_3034 | Gbem_3035 | VgrG | Gbem_3033 | unknown |
| bemidjiensis | | | | | | function |
| (strain Bem/ | | | | | | |
| ATCC BAA- | | | | | | |
| 1014/DSM | | | | | | |
| 16622) | | | | | | |
| Vibrio cholerae | Q9KNE6 | VCA0019 | VCA0018 | VgrG | VCA0020 | Colicin |
| serotype O1 | | | | | | |
| (strain ATCC | | | | | | |
| 39315/El Tor | | | | | | |
| Inaba N16961) | | | | | | |
| | Q9KS44 | VC1417 | VC1416 | VgrG | VC1418 | triacylglycerol |
| | | | | | | lipase |
| Aeromonas | A0KHB0 | AHA_1120 | AHA_1119 | VgrG | AHA_1121 | unknown |
| hydrophila | | | | | | function |
| subsp. | | | | | | |
| hydrophila | | | | | | |
| (strain ATCC | | | | | | |
| 7966/NCIB | | | | | | |
| 9240) | | | | | | |
| Klebsiella | G8VZI3 | KPHS_23050 | KPHS_23040 | VgrG | KPHS_23060 | unknown |
| pneumoniae | | | | | | function |
| subsp. | | | | | | |
| pneumoniae | | | | | | |
| HS11286 | | | | | | |
| Escherichia coli | B7MQ45 | ECED1_0242 | ECED1_0241 | VgrG | ECED1_0244 | unknown |
| O81 (strain | | | | | | function |
| ED1a) | | | | | | |
| Photorhabdus | Q7MZQ6 | plu4221 | plu4222 | VgrG | plu4220 | unknown |
| luminescens | | | | | | function |
| subsp. | | | | | | |
| laumondii | | | | | | |
| (strain TT01) | | | | | | |
| | Q7N252 | plu3245 | plu3246 | VgrG | plu3244 | unknown |
| | | | | | | function |
| | Q7MYS1 | plu4602 | plu4601 | VgrG | plu4603 | Phage_lambda_P |
| | | | | | | Replication |
| | | | | | | protein P |
| | Q7N5M1 | plu1927 | plu1926 | VgrG | plu1928 | unknown |
| | | | | | | function |
| Escherichia coli | D3GS63 | EC042_0228 | EC042_0227 | VgrG | EC042_0229 | unknown |
| O44:H18 | | | | | | function |
| (strain 042/ | | | | | | |
| EAEC) | | | | | | |
| Burkholderia | Q63V54 | BPSL1388 | BPSL1389 | unknown | BPSL1387 | consensus |
| pseudomallei | | | | function | | |
| (strain K96243) | | | | | | |
| | Q63TB8 | BPSL2049 | BPSL2050 | VgrG | BPSL2048A | unknown |
| | | | | | | function |
| | Q63TC9 | BPSL2040 | BPSL2041 | VgrG | BPSL2039 | unknown |
| | | | | | | function |
| | Q63T82 | BPSL2086 | BPSL2085 | unknown | BPSL2087 | unknown |
| | | | | function | | function |

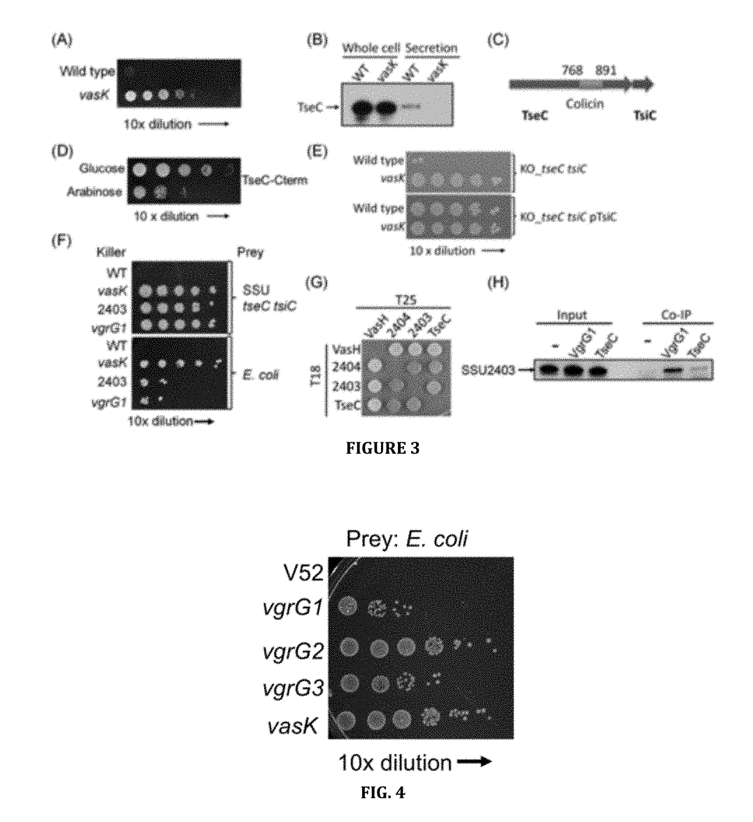

To functionally validate predictive method of the invention, A. hydrophila SSU was used as a model. The T6SS of SSU is known to target eukaryotic cells (42, 48), but T6SS-mediated antibacterial activities have not been demonstrated. To assess the function of T6SS in A. hydrophila, a T6SS null mutant was constructed lacking the vasK gene essential for T6SS functions (42). Using a bacterial killing assay (18), it was found that wild type SSU killed E. coli by 10,000 fold in comparison with the vasK mutant (FIG. 3A), indicating the T6SS of SSU is highly effective in interbacterial competition.

To test whether the predicted effector TseC is secreted by A. hydrophila T6SS (FIG. 2), an epitope-tagged TseC was expressed in the wild type and the vasK mutant. Western blot analysis showed that TseC was secreted only by the wild type but not the vasK mutant (FIG. 3B). TseC is predicted to carry a colicin domain (FIG. 3C). Because colicins attack E. coli through binding to cell membranes (49), TseC antibacterial toxicity was determined by expressing the colicin domain in the periplasm of E. coli using a twin-arginine secretion signal (50, 51). Periplasmic expression of the colicin domain reduced the survival of E. coli to 1% after induction (FIG. 3D) indicating that A. hydrophila TseC is potently antibacterial.

Because previously characterized T6SS-dependent toxic effectors coexist with antagonistic immunity proteins that are encoded by downstream genes (18, 24), it is believed that the gene downstream of tseC is the cognate immunity gene, hereafter referred to as tsiC. If TsiC is the immunity protein to TseC, the tsiC mutant would be susceptible to wild type T6SS-mediated killing by delivery of TseC. This hypothesis was tested by constructing a double knockout mutant lacking both tseC and tsiC. Indeed, the tseC tsiC mutant was efficiently killed by 10⁴-fold when exposed to wild type SSU, and complementation with a plasmid-borne tsiC fully protected the tseC tsiC mutant from killing (FIG. 3E), indicating TsiC is the cognate immunity protein to TseC.

Upstream of SSU tseC are vgrG1 (ORF2404) (48) and the DUF4123 gene ORF2403. It is believed that the secretion of TseC requires VgrG1 and ORF2403 in SSU. To test this, deletion mutants of vgrG1 and ORF2403 were constructed and tested their effects on killing the tseC tsiC double mutant. Neither mutant could kill the tseC tsiC double mutant (FIG. 3F). In contrast, E. coli was efficiently killed by these mutants, indicating that ORF2403 and VgrG1 are not required for T6SS functions. Given the dependency of TseC delivery on ORF2403 and VgrG1, experiments were conducted to determine whether these proteins interact. Using the bacterial two hybrid assay (FIG. 3G) and co-immunoprecipitation (FIG. 3H), the interaction of ORF2403 with VgrG1 and TseC were identified. In addition, the tseC mutant could still kill E. coli, suggesting the existence of other T6SS-dependent antibacterial effectors in SSU (FIG. 5).

Since the discovery of the T6SS in V. cholerae and P. aeruginosa, considerable effort has been made toward understanding the delivery mechanism and the physiological functions (2, 4, 14, 52). Previous research highlights that numerous human pathogens employ the T6SS to deliver toxic effectors to their bacterial competitors or eukaryotic hosts (2, 4). Recent reports on T6SS function in the Bacteroidetes (11) and Agrobacterium (10) further underline the importance of T6SS in dictating bacterial dynamics in complex communities, such as the microbiota in humans and plants. Despite their importance, the identification and assignment of enzymatic function to T6SS effectors still remains challenging. Comparative analysis of effector sequences from different species could be employed to identify potential homologs. However, systematic identification of effectors using bioinformatics is difficult because known T6SS effectors are highly diverse in sequence and function. Although previous studies have successfully identified a number of effectors based on the physical characteristics of known effectors (32) and a N-terminal sequence marker (31), respectively, neither method could identify TseL in V. cholerae.

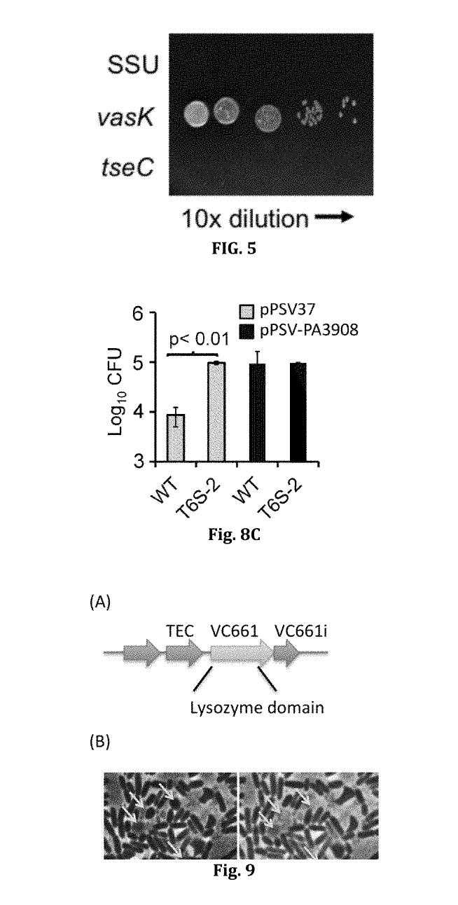

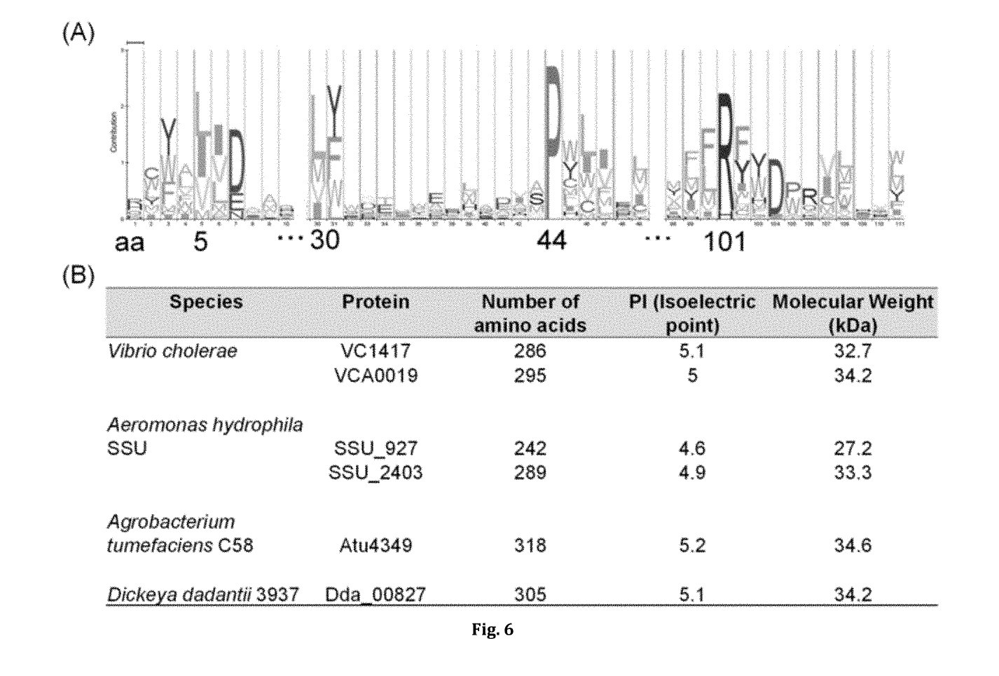

In this study, instead of relying on the diverse effector sequences, the present inventors have demonstrated an effective approach of using a conserved domain (DUF4123) to identify the associated downstream effectors. Results herein show that DUF4123 proteins directly interact with the cognate VgrG and effector proteins and play an essential role in effector delivery, but DUF4123 proteins are not secreted or required for effector activities. DUF4123 thus appears to function similarly to the chaperone proteins of T4 phage, gp38 (53, 54) and gp63 (55, 56), which are important for tail fiber assembly and attachment but are not components of the mature phage particle (57). In addition, the secretion of many effectors of the type 3 secretion (TTS) system is dependent on specific interaction with cognate chaperone proteins that are present in the cytosol but not secreted (58, 59). Interestingly, TTS chaperone proteins generally have low molecular weight and acidic isoelectric point (PI<5), and the chaperone genes are often found next to the genes encoding cognate effectors (58, 59). It was found that DUF4123 proteins also exhibit low PI values (˜5) and the domain has several highly conserved residues (FIG. 6). Because of the abovementioned characteristics of DUF4123 proteins, it is believed that DUF4123 proteins function as T6SS effector chaperones (TEC) and thus name the DUF4123 domain TEC, the VC1417 protein TecL, and the SSU2403 protein TecC.

TEC genes are widely distributed in Proteobacteria and are largely located together with an upstream VgrG/PAAR gene. It is believed that downstream of TEC genes are genes encoding candidate T6SS effectors. Using the TEC sequence, this theory was validated by identifying known effectors, including TseL and VasX in V. cholerae that share few common features in sequence, function, and structure. Using the method of the invention, a new T6SS dependent effector-immunity pair TseC-TsiC in the A. hydrophila SSU strain were discovered.

There are two models of mechanism for T6SS protein export. The first model requires effectors bind to the inner surface of the ring-like Hcp hexamers (52) while the second, termed Multiple Effector Translocation VgrG (MERV), involves binding of effectors to the tip VgrG and PAAR proteins (2, 14, 18). The limited inner space of the Hcp hexameric ring likely poses a physical restraint on the size of effectors relying on binding to Hcp as chaperones for delivery (4). In the MERV model, binding to the tip proteins renders more flexibility to accommodate effectors that differ greatly in size and sequence (2, 14). Indeed, a number of effectors with diverse functions have been reported to require VgrG and PAAR proteins for delivery (14, 18, 41). Results provided herein on the VgrG-dependent secretion of TseL in V. cholerae and TseC in A. hydrophila further support the MERV model.

Many bacterial species possess multiple TEC proteins. For example, A. hydrophila SSU has two TEC proteins (FIG. 2). These two TEC proteins cannot functionally complement each other, as evidenced by the loss of killing resulting from deletion in tecC (ORF2403) (FIG. 3F). Because TEC proteins interact with both conserved VgrG proteins and divergent effectors, we propose that the conserved TEC domain is responsible for binding to VgrG/PAAR while each TEC protein has acquired specific sequences to accommodate binding to its partner effector. For T6SS-mediated delivery of a given VgrG-binding effector, multiple binding events likely occur in a temporal order that includes effector binding to the cognate VgrG, to the TEC protein, and to the immunity protein (if the immunity protein is present in the cytosol). Since TEC proteins and immunity proteins are not secreted, the separation of effectors from the cognate TEC and immunity proteins probably takes place prior to binding to VgrG for delivery. It is possible that TEC proteins coordinate the process of effector loading to the VgrG/PAAR spike to prevent premature binding of effectors with VgrG. The formation of T6SS spike might expose the effector-binding site of VgrG that attracts effectors and displaces TEC proteins. Because TEC proteins can bind to both effectors and VgrGs, it is also possible that TEC proteins facilitate the binding of VgrG and effectors by presenting the binding partners in right conformation or maintaining protein stability. Structural analyses of TEC, VgrG and effector proteins are required to fully understand not only the actions of TEC but also the mechanisms of T6SS effector delivery.

Provided herein is a new class of diverse and TEC-dependent T6SS effectors. Methods disclosed herein can be used for characterizing the functions of these effectors in different model systems which will greatly increase the understanding of the physiological role T6SS plays in these species. Given the diverse ecological niches these species occupy in the environment and the host, more novel functions of T6SS effectors are likely to be discovered using the method of the invention. Because T6SS effectors are known to target essential cellular functions including the cell wall, membrane, and DNA/RNA of bacteria and the membrane and cytoskeleton of eukaryotic cells (2, 4), the toxicity of effectors may provide an alternative therapeutic approach of treating bacterial infections or killing specific types of eukaryotic cells.

Additional objects, advantages, and novel features of this invention will become apparent to those skilled in the art upon examination of the following examples thereof, which are not intended to be limiting. In the Examples, procedures that are constructively reduced to practice are described in the present tense, and procedures that have been carried out in the laboratory are set forth in the past tense.

### Examples

Strains and plasmids used in this study are listed in Table B. Cultures were routinely grown aerobically at 37° C. in LB (w/v 1% tryptone, 0.5% yeast extract, and 0.5% NaCl). Antibiotics and chemicals were used at the following concentrations: ampicillin (100 μg/ml), streptomycin (100 μg/ml), kanamycin (50 μg/ml), tetracycline (10 μg/ml), chloramphenicol (25 μg/ml for E. coli, 2.5 μg/ml for SSU and V52), IPTG (1 mM) and arabinose (w/v 0.1%). Mutants of SSU and V52 were constructed using crossover PCR and homologous recombination (60, 61). Gene expression vectors were constructed as previously described (38). All constructs were verified by sequencing.

TABLE B
Strains and plasmids in this study.

| Strain and plasmid | Genotype or phenotype | Reference |
|---|---|---|
| V. cholerae V52 | | |
| wild type | Serotype 37 clinical isolate from Sudan | (1) |
| vasK | A T6SS null mutant lacking the vasK gene | (1) |
| VC1417-21 | V52 lacking VC1417-21 | (2) |
| vgrG1 | deletion mutant of VC1416 | (2) |
| vgrG2 | deletion mutant of VCA0018 | (2) |
| vgrG3 | deletion mutant of VCA0123 | (2) |
| A. hydrophila SSU | | |
| wild type | A diarrheal isolate | (3) |
| vask | T6SS null mutant, in frame deletion of vasK | this study |
| tseC | deletion mutant of ORF2402 | this study |
| vgrG1 | deletion mutant of ORF2404 | this study |
| ORF2403 | deletion mutant of ORF2403 | this study |
| tseC tsiC | deletion mutant of ORF2402-2401 | this study |
| E. coli | | |
| SM10 | thi thr leu tonA lac Y supE recA::RP4-2-Tc::Mu | (4) |
| BTH101 | F−, cya-99, araD139, galE15, galK16, rpsL1 (Strr), hsdR2, | (5) |
| | mcrA1, mcrB1 | |
| MG1655gen | A random transposon mutant used as prey for T6SS killing | (6) |
| CC114 | a tetracycline-resistant E. coli used for T6SS killing | (2) |
| DH5alpha | F− endA1 glnV44 thi-1 recA1 relA1 gyrA96 deoR nupG | New England |
| | Φ80dlacZΔM15 Δ(lacZYA-argF)U169, hsdR17(rK− mK+), λ− | Biolabs |
| BL21DE3 | F− ompT gal dcm lon hsdSB(rB− mB−) λ(DE3 [lacI lacUV5-T7 | New England |
| | gene 1 ind1 sam7 nin5]) | Biolabs |
| Plasmid | | |
| pWM91 | Suicidal conjugation vector for making in-frame deletion | (7) |
| pDS132 | Suicidal conjugation vector for making in-frame deletion | (2) |
| pBAD18V5 | Arabinose-inducible expression vector with 3xV5 tag | (8) |
| pBAD18kan | Arabinose-inducible expression vector | (9) |
| pBAD24 | Arabinose-inducible expression vector | (9) |
| pETDuet | IPTG-inducible expression vector | Novagen |
| pACYCDuet | IPTG-inducible expression vector | Novagen |
| pKNT25 | T25 adenylate cyclase domain, KanR | (5) |
| pCH363 | T18 adenylate cylcase domain, AmpR | (5) |

(1) Pukatzki S, et al. (2006) Identification of a conserved bacterial protein secretion system in Vibrio cholerae using the Dictyostelium host model system. Proc Natl Acad Sci USA 103(5): 1528-1533.
(2) Zheng J, Ho B, Mekalanos J J (2011) Genetic analysis of anti-amoebae and anti-bacterial activities of the type VI secretion system in Vibrio cholerae. PloS One 6(8): e23876.
(3) Suarez G, et al. (2008) Molecular characterization of a functional type VI secretion system from a clinical isolate of Aeromonas hydrophila. Microb Pathog 44(4): 344-61.
(4) Miller V L, Mekalanos J J (1988) A novel suicide vector and its use in construction of insertion mutations: osmoregulation of outer membrane proteins and virulence determinants in Vibrio cholerae requires toxR. J Bacteriol 170(6): 2575-2583.
(5) Karimova G, Pidoux J, Ullmann A, Ladant D (1998) A bacterial two-hybrid system based on a reconstituted signal transduction pathway. Proc Natl Acad Sci USA 95(10): 5752-5756.
(6) Basler M, Ho BT, Mekalanos JJ (2013) Tit-for-tat: type VI secretion system counterattack during bacterial cell-cell interactions. Cell 152(4): 884-894.
(7) Metcalf W W, et al. (1996) Conditionally replicative and conjugative plasmids carrying lacZ alpha for cloning, mutagenesis, and allele replacement in bacteria. Plasmid 35(1): 1-13.
(8) Davies B W, Bogard R W, Mekalanos J J (2011) Mapping the regulon of Vibrio cholerae ferric uptake regulator expands its known network of gene regulation. Proc Natl Acad Sci USA 108(30): 12467-12472.
(9) Guzman L M, Belin D, Carson M J, Beckwith J (1995) Tight regulation, modulation, and high-level expression by vectors containing the arabinose PBAD promoter. J Bacteriol 177(14): 4121-4130.

Western Blotting Analysis:

Protein samples were loaded on a precast 4-12% SDS-PAGE gel (Life Technologies), run at 180V for 40 min and transferred to a PVDF membrane (Millipore) by electrophoresis. The membrane was blocked with 5% non-fat milk in TBST buffer (50 mM Tris, 150 mM NaCl, 0.05% Tween-20, pH7.6) for 1 hour at room temperature, incubated with primary antibodies at 4° C. overnight, washed three times in TBST buffer, and incubated with a HRP-conjugated secondary antibody (Cell Signaling Technology) for 1 hour followed by detection using the ECL solution (Bio-Rad) and a ChemiDoc MP system (Bio-Rad). The monoclonal antibodies to epitope tags, anti-V5, anti-FLAG, and anti-6xHIS were purchased from Sigma Aldrich. The monoclonal antibody to RpoB, the beta subunit of RNA polymerase, was purchased from NeoClone and used as a loading control for western blot analysis as previously described (62).

Protein Secretion Assay:

Exponential phase cultures (OD₆₀₀=0.5) grown in LB were induced by adding 0.1% L-arabinose for 1 hour. One ml culture was collected by centrifugation twice at 20,000×g for 2 min and then filtered through a 0.2-μm filter. The filtered supernatant was combined with 200 μl of 100% ice-cold TCA solution, placed on ice for 2 hours and centrifuged at 15,000×g for 30 min at 4° C. The pellet was washed with 1 ml of 100% acetone by centrifugation at 20,000×g for 5 min, air-dried and mixed with 30 μl of SDS-loading dye, followed by SDS-PAGE and western blot analyses as described above.

Bacterial Cell Killing Assay:

Killing assay was performed as previously described (33). Briefly, cultures were mixed together at a ratio of 10:1 (predator to prey), spotted on LB medium for 3 hours at 37° C., and then resuspended in 1 ml of LB. Survival of prey cells was quantified by serial dilution in LB and plating on selective medium.

Bacterial Two-Hybrid Assay:

The two-hybrid assay was performed as described (34, 63). Plasmid vectors carrying the indicated T18 and T25 constructs were transformed to BTH101 (cya-99). Individual colonies were grown in LB for 3 hours and then patched on LB medium supplemented with Amp, Kan, X-Gal, and 0.5 mM IPTG. Plates were incubated at room temperature for at least 48 hours.

Co-Immunoprecipitation Assay:

Genes were cloned into pETDuet-1 and pACYCDuet-1 vectors for expression. E. coli BL21DE3 carrying different gene expression vectors were grown in 50 ml LB culture to exponential phase OD600=0.5, and induced by 1 mM IPTG for 3 hours at 37° C. Cells were collected by centrifugation at 4,000 g for 10 min and resuspended in 5 ml PBS buffer. Cell lysates were prepared by sonication and cell debris were removed by centrifugation at 20,000 g for 20 min. Dynabeads Protein G (Life Technologies) were incubated with 5 μg of monoclonal antibodies to 6His or V5 for 2 hours at 4° C., and then mixed with 1 ml cell lysates for 3 hours at 4° C. The magnetic beads were washed three times with ice-cold PBST (phosphate saline buffer with v/v 0.2% Tween-20) and then incubated with 30 μl of SDS-loading buffer, followed by incubation at 70° C. for 10 min to elute bound proteins. Eluted samples were subject to western blotting analysis as described above.

Bioinformatic Analysis:

Protein sequences were retrieved from NCBI database, and analyzed using HHpred (43) and Phyre2 (44, 45) for functional prediction. Representative DUF4123 protein accession numbers and species were downloaded from the Pfam protein database (39). Species carrying the DUF4123 domain, VipA (DFU770) and Hcp (DUF796) were downloaded from the Interpro database and compared using the Gene List Venn Diagram program (http://genevenn.sourceforge.net/). Using the Pfam generated species tree of DUF4123, we selected representative species from each genus with fully annotated genomes to characterize the DUF4123 immediate upstream and downstream proteins using the protein annotation in the NCBI database.

Complementation with Immunity Genes Confers Protection:

FIG. 7B shows test results confirming of the TEC-dependent effector SSU928 and its immunity SSU928i in Aeromonas hydrophila. Mutants, Δ928ei (schematically illustrated in FIG. 7A) and Δ947ei, carrying an empty pBAD18Kan vector or the immunity genes, were mixed with wild type SSU and the vasK mutant at a ratio of 1:2 and incubated for 3 hours. Survival of the mutants was enumerated by serial dilutions. The ΔvasK mutant lacks an essential membrane component of the type VI protein secretion system and thus cannot deliver effector proteins. As shown in FIG. 7(B), results indicate that SSU928 is a highly effective antimicrobial effector and its immunity protein SSU928i confers protection against SSU928 toxicity.

Complementation of the Double Mutant with PA3908 Restores Survival:

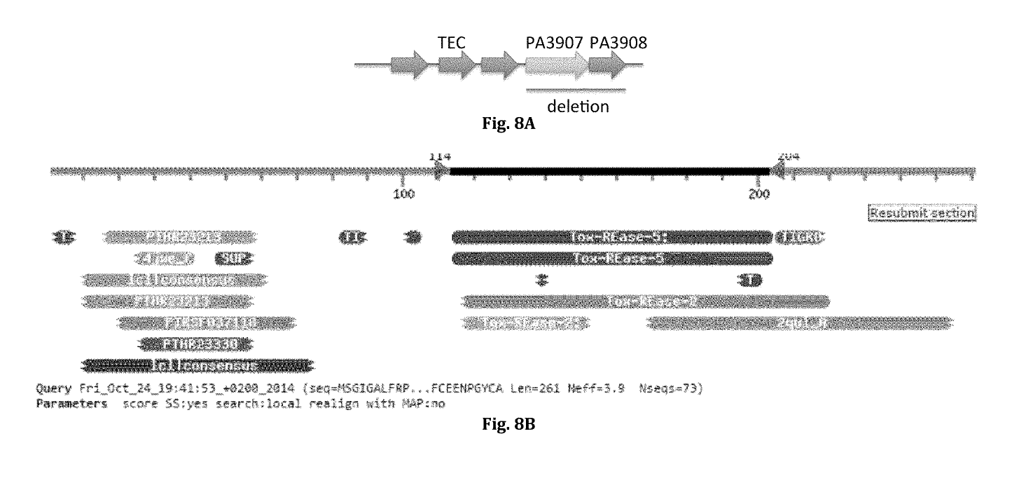

FIGS. 8A-8C confirms the TEC-dependent effector PA3907 and its immunity protein PA3908 in Pseudomonas aeruginosa. Using the operon structure of the PA3907 gene cluster, as shown in FIG. 8A, a deletion mutant lacking both PA3907 and PA3908 was constructed. As shown in FIG. 8B, PA3907 possess a conserved toxic Tox-REase-5 functional domain. As the test results shown in FIG. 8C indicate, the deletion mutant of PA3907 and PA3908 was efficiently killed by wild type Pseudomonas aeruginosa but not by the H2-T6SS cluster mutant T6S-2. These results indicate that PA3908 is the immunity protein that confers protection against PA3907 effector toxicity.

Identification of a Novel Effector-Immunity Pair in Vibrio cholera:

As illustrated in FIG. 9 panel (A), operon structure of the effector VC661 and it immunity gene VC661i are located near to one another. VC661 carries a predicted lysozyme domain targeting the cell wall. As shown in FIG. 9 panel (B), expression of VC661 in the periplasm of E. coli results in extensive cell lysis, indicating severe damage to the cell wall.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter. All references cited herein are incorporated by reference in their entirety.

### CITED REFERENCES

1. Fronzes R, Christie P J, Waksman G (2009) The structural biology of type IV secretion systems. Nat Rev Microbiol 7(10):703-14.
2. Ho B T, Dong T G, Mekalanos J J (2014) A view to a kill: the bacterial type VI secretion system. Cell Host Microbe 15(1):9-21.
3. Hood R D, et al. (2010) A type VI secretion system of Pseudomonas aeruginosa targets a toxin to bacteria. Cell Host Microbe 7(1):25-37.
4. Russell A B, Peterson S B, Mougous J D (2014) Type VI secretion system effectors: poisons with a purpose. Nat Rev Microbiol 12(2):137-48.
5. Leiman P G, et al. (2009) Type VI secretion apparatus and phage tail-associated protein complexes share a common evolutionary origin. Proc Natl Acad Sci USA 106(11):4154-4159.
6. Pukatzki S, et al. (2006) Identification of a conserved bacterial protein secretion system in Vibrio cholerae using the Dictyostelium host model system. Proc Natl Acad Sci USA 103(5):1528-1533.
Mougous J D, et al. (2006) A virulence locus of Pseudomonas aeruginosa encodes a protein secretion apparatus. Science 312(5779):1526-1530.
8. Bingle L E, Bailey C M, Pallen M J (2008) Type VI secretion: a beginner's guide. Curr Opin Microbiol 11(1):3-8.
9. Fu Y, Waldor M K, Mekalanos J J (2013) Tn-Seq Analysis of Vibrio cholerae Intestinal Colonization Reveals a Role for T6SS-Mediated Antibacterial Activity in the Host. Cell Host Microbe 14(6):652-663.
10. Ma L-S, Hachani A, Lin J-S, Filloux A, Lai E-M (2014) Agrobacterium tumefaciens Deploys a Superfamily of Type VI Secretion DNase Effectors as Weapons for Interbacterial Competition In Planta. Cell Host Microbe 16(1):94-104.
11. Russell A B, et al. (2014) A type VI secretion-related pathway in Bacteroidetes mediates interbacterial antagonism. Cell Host Microbe 16(2):227-236.
12. Kudryashev M, et al. (2015) Structure of the Type VI Secretion System Contractile Sheath. Cell 160(5):952-962.
13. Basler M, Pilhofer M, Henderson G P, Jensen G J, Mekalanos J J (2012) Type VI secretion requires a dynamic contractile phage tail-like structure. Nature 483(1476-4687 (Electronic)):182-186.
14. Shneider M M, et al. (2013) PAAR-repeat proteins sharpen and diversify the type VI secretion system spike. Nature 500(7462):350-353.
15. Basler M, Mekalanos J J (2012) Type 6 secretion dynamics within and between bacterial cells. Science 337(6096):815.
16. Bönemann G, Pietrosiuk A, Diemand A, Zentgraf H, Mogk A (2009) Remodelling of VipA/VipB tubules by ClpV-mediated threading is crucial for type VI protein secretion. EMBO J 28(4):315-325.
17. Filloux A, Hachani A, Bleves S (2008) The bacterial type VI secretion machine: yet another player for protein transport across membranes. Microbiol Read Engl 154(Pt 6):1570-1583.
18. Dong T G, Ho B T, Yoder-Himes D R, Mekalanos J J (2013) Identification of T6SS-dependent effector and immunity proteins by Tn-seq in Vibrio cholerae. Proc Natl Acad Sci USA 110:2623-8.
19. Brooks T M, Unterweger D, Bachmann V, Kostiuk B, Pukatzki S (2013) Lytic activity of the Vibrio cholerae type VI secretion toxin VgrG-3 is inhibited by the antitoxin TsaB. J Biol Chem 288(11):7618-7625.
20. Koskiniemi S, et al. (2013) Rhs proteins from diverse bacteria mediate intercellular competition. Proc Natl Acad Sci USA 110(17):7032-7.
21. Pukatzki S, Ma A T, Revel A T, Sturtevant D, Mekalanos J J (2007) Type VI secretion system translocates a phage tail spike-like protein into target cells where it cross-links actin. Proc Natl Acad Sci USA 104(39):15508-15513.
22. Suarez G, et al. (2010) A type VI secretion system effector protein, VgrG1, from Aeromonas hydrophila that induces host cell toxicity by ADP ribosylation of actin. J Bacteriol 192(1):155-68.
23. Miyata S T, Unterweger D, Rudko S P, Pukatzki S (2013) Dual expression profile of type VI secretion system immunity genes protects pandemic Vibrio cholerae. PLoS Pathog 9(12):e1003752.
24. Russell A B, et al. (2011) Type VI secretion delivers bacteriolytic effectors to target cells. Nature 475(7356):343-347.
25. Dong T G, et al. (2015) Generation of reactive oxygen species by lethal attacks from competing microbes. Proc Natl Acad Sci USA 112(7):2181-2186.
26. Dwyer D J, et al. (2014) Antibiotics induce redox-related physiological alterations as part of their lethality. Proc Natl Acad Sci:201401876.
27. Kohanski M A, Dwyer D J, Hayete B, Lawrence C A, Collins J J (2007) A common mechanism of cellular death induced by bactericidal antibiotics. Cell 130(5):797-810.
28. Whitney J C, et al. (2014) Genetically distinct pathways guide effector export through the type VI secretion system. Mol Microbiol 92(3):529-542.
29. Altindis E, Dong T, Catalano C, Mekalanos J (2015) Secretome Analysis of Vibrio cholerae Type VI Secretion System Reveals a New Effector-Immunity Pair. mBio 6(2):e00075-15.
30. English G, et al. (2012) New secreted toxins and immunity proteins encoded within the Type VI secretion system gene cluster of Serratia marcescens. Mol Microbiol 86(4):921-936.
31. Salomon D, et al. (2014) Marker for type VI secretion system effectors. Proc Natl Acad Sci USA 111(25):9271-9276.
32. Russell A B, et al. (2012) A widespread bacterial type VI secretion effector superfamily identified using a heuristic approach. Cell Host Microbe 11(5):538-549.
33. Zheng J, Ho B, Mekalanos J J (2011) Genetic analysis of anti-amoebae and anti-bacterial activities of the type VI secretion system in Vibrio cholerae. PloS One 6(8):e23876.
34. Karimova G, Pidoux J, Ullmann A, Ladant D (1998) A bacterial two-hybrid system based on a reconstituted signal transduction pathway. Proc Natl Acad Sci USA 95(10):5752-5756.
35. Ma A T, McAuley S, Pukatzki S, Mekalanos J J (2009) Translocation of a Vibrio cholerae type VI secretion effector requires bacterial endocytosis by host cells. Cell Host Microbe 5(3):234-243.
36. Kitaoka M, Miyata S T, Brooks T M, Unterweger D, Pukatzki S (2011) VasH is a transcriptional regulator of the type VI secretion system functional in endemic and pandemic Vibrio cholerae. J Bacteriol 193(23):6471-6482.
37. Bernard C S, Brunet Y R, Gavioli M, Lloubés R, Cascales E (2011) Regulation of type VI secretion gene clusters by sigma54 and cognate enhancer binding proteins. J Bacteriol 193(9):2158-67.
38. Dong T G, Mekalanos J J (2012) Characterization of the RpoN regulon reveals differential regulation of T6SS and new flagellar operons in Vibrio cholerae 037 strain V52. Nucleic Acids Res 40:7766-75.
39. Finn R D, et al. (2014) Pfam: the protein families database. Nucleic Acids Res 42(D1):D222-D230.
40. Mougous J D, Gifford C A, Ramsdell T L, Mekalanos J J (2007) Threonine phosphorylation post-translationally regulates protein secretion in Pseudomonas aeruginosa. Nat Cell Biol 9(7):797-803.
41. Hachani A, Allsopp L P, Oduko Y, Filloux A (2014) The VgrG proteins are “á la carte” delivery systems for bacterial type VI effectors. J Biol Chem 289(25):17872-17884.
42. Suarez G, et al. (2008) Molecular characterization of a functional type VI secretion system from a clinical isolate of Aeromonas hydrophila. Microb Pathog 44(4):344-61.
43. Söding J, Biegert A, Lupas A N (2005) The HHpred interactive server for protein homology detection and structure prediction. Nucleic Acids Res 33(Web Server issue):W244-248.
44. Kelley L A, Sternberg M J E (2009) Protein structure prediction on the Web: a case study using the Phyre server. Nat Protoc 4(3):363-71.
45. Kelley L A, Mezulis S, Yates C M, Wass M N, Sternberg M J E (2015) The Phyre2 web portal for protein modeling, prediction and analysis. Nat Protoc 10(6):845-858.
46. Meusch D, et al. (2014) Mechanism of Tc toxin action revealed in molecular detail. Nature 508(7494):61-65.
47. Waterfield N R, Bowen D J, Fetherston J D, Perry R D, ffrench-Constant R H (2001) The tc genes of Photorhabdus: a growing family. Trends Microbiol 9(4):185-191.
48. Sha J, et al. (2013) Evaluation of the roles played by Hcp and VgrG type 6 secretion system effectors in Aeromonas hydrophila SSU pathogenesis. Microbiol Read Engl 159(Pt 6):1120-1135.
49. Cascales E, et al. (2007) Colicin biology. Microbiol Mol Biol Rev MMBR 71(1):158-229.
50. Palmer T, Berks B C (2012) The twin-arginine translocation (Tat) protein export pathway. Nat Rev Microbiol 10(7):483-96.
51. Berks B C (1996) A common export pathway for proteins binding complex redox cofactors? Mol Microbiol 22(3):393-404.
52. Silverman J M, et al. (2013) Haemolysin coregulated protein is an exported receptor and chaperone of type VI secretion substrates. Mol Cell 51(5):584-93.
53. King J, Wood W B (1969) Assembly of bacteriophage T4 tail fibers: the sequence of gene product interaction. J Mol Biol 39(3):583-601.
54. Bishop R J, Wood W B (1976) Genetic analysis of T4 tail fiber assembly. I. A gene 37 mutation that allows bypass of gene 38 function. Virology 72(1):244-254.
55. Wood W B, Conley M P, Lyle H L, Dickson R C (1978) Attachment of tail fibers in bacteriophage T4 assembly. Purification, properties, and site of action of the accessory protein coded by gene 63. J Biol Chem 253(7):2437-2445.
56. Snopek T J, Wood W B, Conley M P, Chen P, Cozzarelli N R (1977) Bacteriophage T4 RNA ligase is gene 63 product, the protein that promotes tail fiber attachment to the baseplate. Proc Natl Acad Sci 74(8):3355-3359.
57. Vanderslice R W, Yegian C D (1974) The identification of late bacteriophage T4 proteins on sodium dodecyl sulfate polyacrylamide gels. Virology 60(1):265-275.
58. Page A-L, Parsot C (2002) Chaperones of the type III secretion pathway: jacks of all trades. Mol Microbiol 46(1):1-11.
59. Ghosh P (2004) Process of protein transport by the type III secretion system. Microbiol Mol Biol Rev MMBR 68(4):771-795.
60. Miller V L, Mekalanos J J (1988) A novel suicide vector and its use in construction of insertion mutations: osmoregulation of outer membrane proteins and virulence determinants in Vibrio cholerae requires toxR. J Bacteriol 170(6):2575-2583.
61. Philippe N, Alcaraz J-P, Coursange E, Geiselmann J, Schneider D (2004) Improvement of pCVD442, a suicide plasmid for gene allele exchange in bacteria. Plasmid 51(3):246-55.
62. Dong T, Yu R, Schellhorn H (2011) Antagonistic regulation of motility and transcriptome expression by RpoN and RpoS in Escherichia coli. Mol Microbiol 79(2):375-386.
63. Yang D C, et al. (2011) An ATP-binding cassette transporter-like complex governs cell-wall hydrolysis at the bacterial cytokinetic ring. Proc Natl Acad Sci USA 108(45):E1052-1060.

What is claimed is:

1. A method for identifying a type VI secretion system (“T6SS”) effector in Gram-negative bacteria, said method comprising:
(a) identifying a conserved domain sequence of T6SS of Gram-negative bacteria;
(b) searching upstream and downstream of said conserved domain sequence of T6SS of said Gram-negative bacteria;
(c) producing a mutant type of said Gram-negative bacteria by deleting a gene upstream or downstream from said conserved domain sequence of T6SS, wherein said deleted gene is within ten gene sequences from said conserved domain sequence of T6SS;
(d) determining the antibacterial activity of said mutant compared to a wild-type; and
(e) identifying a T6SS effector based on the observed antimicrobial activity of said mutant and said wild-type.

2. The method of claim 1, wherein prior to said step (e), said method further comprises repeating said steps (c) and (d) until a difference in the antibacterial activity between said mutant and said wild-type is observed.

3. The method of claim 1, wherein said conserved domain sequence of T6SS comprises VC1417 gene.

4. The method of claim 3, wherein said conserved domain sequence of T6SS comprises a conserved domain DUF4123.

5. The method of claim 1 further comprising the step of identifying a T6SS effector immunity protein, wherein said method of identifying a T6SS effector immunity protein comprises:
(i) producing a second mutant type of said Gram-negative bacteria by deleting a gene upstream or downstream from said conserved domain sequence of T6SS, wherein said deleted gene is within ten gene sequences from said conserved domain sequence of T6SS;
(ii) culturing said second mutant type in the presence of said wild-type; and
(iii) identifying a T6SS effector immunity protein based on the survival of said mutant type in the presence of said wild-type.

6. A method for treating bacterial infection in a subject comprising administering to a subject in need of bacterial infection treatment a therapeutically effective amount of a composition comprising a type VI secretion system (T6SS) effector discovered using a method of claim 1.

7. The method of claim 6, wherein said T6SS effector comprises a protein that is encoded by a gene listed in Table 1 or Table 2.

8. A method for treating a Gram-negative bacteria infection in a subject, said method comprising administering to a subject in need of Gram-negative bacterial infection treatment a therapeutically effective amount of a composition comprising a type VI secretion system (T6SS) effector immunity protein discovered using a method of claim 5.

9. The method of claim 8, wherein said T6SS effector immunity protein comprises a protein that is encoded by a gene listed in Table 3.

10. A recombinant vector, comprising a gene from Table 1 or Table 2 or Table 3, wherein the gene is operatively linked to a heterologous regulatory sequence.

11. A host cell containing the oligonucleotide of claim 10.

12. A method for delivering a T6SS effector, said method comprising using a T6SS effector chaperone (TEC) protein to deliver a T6SS effector.
